SEQUENCE LISTING (21 sequences)

SEQ ID NO: 1 - DNA, 2685 bp, Staphylococcus aureus
atgaacaaacatcacccaaaattaaggtctttctattctattagaaaatcaactctaggcgttgcatcggtcattgtcagtacactatttttaattacttctcaacatcaagcacaagcagcagaaaatacaaatacttcagataaaatctcggaaaatcaaaataataatgcaactacaactcagccacctaaggatacaaatcaaacacaacctgctacgcaaccagcaaacactgcgaaaaactatcctgcagcggatgaatcacttaaagatgcaattaaagatcctgcattagaaaataaagaacatgatataggtccaagagaacaagtcaatttccagttattagataaaaacaatgaaacgcagtactatcactttttcagcatcaaagatccagcagatgtgtattacactaaaaagaaagcagaagttgaattagacatcaatactgcttcaacatggaagaagtttgaagtctatgaaaacaatcaaaaattgccagtgagacttgtatcatatagtcctgtaccagaagaccatgcctatattcgattcccagtttcagatggcacacaagaattgaaaattgtttcttcgactcaaattgatgatggagaagaaacaaattatgattatactaaattagtatttgctaaacctatttataacgatccttcacttgtaaaatcagatacaaatgatgcagtagtaacgaatgatcaatcaagttcagtcgcaagtaatcaaacaaacacgaatacatctaatcaaaatacatcaacgatcaacaatgctaataatcaaccgcaggcaacgaccaatatgagtcaacctgcacaaccaaaatcgtcaacgaatgcagatcaagcgtcaagccaaccagctcatgaaacaaattctaatggtaatactaacgataaaacgaatgagtcaagtaatcagtcggatgttaatcaacagtatccaccagcagatgaatcactacaagatgcaattaaaaacccggctatcatcgataaagaacatacagctgataattggcgaccaattgattttcaaatgaaaaatgataaaggtgaaagacagttctatcattatgctagtactgttgaaccagcaactgtcatttttacaaaaacaggaccaataattgaattaggtttaaagacagcttcaacatggaagaaatttgaagtttatgaaggtgacaaaaagttaccagtcgaattagtatcatatgattctgataaagattatgcctatattcgtttcccagtatctaatggtacgagagaagttaaaattgtgtcatctattgaatatggtgagaacatccatgaagactatgattatacgctaatggtctttgcacagcctattactaataacccagacgactatgtggatgaagaaacatacaatttacaaaaattattagctccgtatcacaaagctaaaacgttagaaagacaagtttatgaattagaaaaattacaagagaaattgccagaaaaatataaggcggaatataaaaagaaattagatcaaactagagtagagttagctgatcaagttaaatcagcagtgacggaatttgaaaatgttacacctacaaatgatcaattaacagatttacaagaagcgcattttgttgtttttgaaagtgaagaaaatagtgagtcagttatggacggctttgttgaacatccattctatacagcaactttaaatggtcaaaaatatgtagtgatgaaaacaaaggatgacagttactggaaagatttaattgtagaaggtaaacgtgtcactactgtttctaaagatcctaaaaataattctagaacgctgattttcccatatatacctgacaaagcagtttacaatgcgattgttaaagtcgttgtggcaaacattggttatgaaggtcaatatcatgtcagaattataaatcaggatatcaatacaaaagatgatgatacatcacaaaataacacgagtgaaccgctaaatgtacaaacaggacaagaaggtaaggttgctgatacagatgtagctgaaaatagcagcactgcaacaaatcctaaagatgcgtctgataaagcagatgtgatagaaccagagtctgacgtggttaaagatgctgataataatattgataaagatgtgcaacatgatgttgatcatttatccgatatgtcggataataatcacttcgataaatatgatttaaaagaaatggatactcaaattgccaaagatactgatagaaatgtggataaagatgccgataatagcgttggtatgtcatctaatgtcgatactgataaagactctaataaaaataaagacaaagtcatacagctgaatcatattgccgataaaaataatcatactggaaaagcagcaaagcttgacgtagtgaaacaaaattataataatacagacaaagttactgacaaaaaaacaactgaacatctgccgagtgatattcataaaactgtagataaaacagtgaaaacaaaagaaaaagccggcacaccatcgaaagaaaacaaacttagtcaatctaaaatgctaccaaaaactggagaaacaacttcaagccaatcatggtggggcttatatgcgttattaggtatgttagctttattcattcctaaattcagaaaagaatctaaa

SEQ ID NO: 2 - protein, 895 aa, Staphylococcus aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNTSTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 3 - DNA, 431 bp, Staphylococcus aureus
gcggatgaatcacttaaagatgcaattaaagatcctgcattagaaaataaagaacatgatataggtccaagagaacaagtcaatttccagttattagataaaaacaatgaaacgcagtactatcactttttcagcatcaaagatccagcagatgtgtattacactaaaaagaaagcagaagttgaattagacatcaatactgcttcaacatggaagaagtttgaagtctatgaaaacaatcaaaaattgccagtgagacttgtatcatatagtcctgtaccagaagaccatgcctatattcgattcccagtttcagatggcacacaagaattgaaaattgtttcttcgactcaaattgatgatggagaagaaacaaattatgattatactaaattagtatttgctaaacctatttataacgatccttcact

SEQ ID NO: 4 - protein, 144 aa, Staphylococcus aureus
ADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSL

SEQ ID NO: 5 - DNA, 429 bp, Staphylococcus aureus
gcagatgaatcactacaagatgcaattaaaaacccggctatcatcgataaagaacatacagctgataattggcgaccaattgattttcaaatgaaaaatgataaaggtgaaagacagttctatcattatgctagtactgttgaaccagcaactgtcatttttacaaaaacaggaccaataattgaattaggtttaaagacagcttcaacatggaagaaatttgaagtttatgaaggtgacaaaaagttaccagtcgaattagtatcatatgattctgataaagattatgcctatattcgtttcccagtatctaatggtacgagagaagttaaaattgtgtcatctattgaatatggtgagaacatccatgaagactatgattatacgctaatggtctttgcacagcctattactaataacccagacgac

SEQ ID NO: 6 - protein, 143 aa, Staphylococcus aureus
ADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDD

SEQ ID NO: 7 - DNA, 19 bp, Staphylococcus aureus
gatattgataattattatc

SEQ ID NO: 8 - DNA, 36 bp, Artificial Sequence
  Description of Artificial Sequence  Synthetic Primer
aggcatggtctctgcgccgcagaaaatacaaatact

SEQ ID NO: 9 - DNA, 36 bp, Artificial Sequence
  Description of Artificial Sequence  Synthetic Primer
attgctggtctcttatcaagtttttggtagcatttt

SEQ ID NO: 10 - DNA, 32 bp, Artificial Sequence
  Description of Artificial Sequence  Synthetic Primer
aatggatccgcggatgaatcacttaaagatgc

SEQ ID NO: 11 - DNA, 36 bp, Artificial Sequence
  Description of Artificial Sequence  Synthetic Primer
tatgtcgacctaaagtgaaggatcgttataaatagg

SEQ ID NO: 12 - DNA, 32 bp, Artificial Sequence
  Description of Artificial Sequence  Synthetic Primer
aatggatccgcagatgaatcactacaagatgc

SEQ ID NO: 13 - DNA, 36 bp, Artificial Sequence
  Description of Artificial Sequence  Synthetic Primer
tatgtcgacctagtcgtctgggttattagtaatagg

SEQ ID NO: 14 - DNA, 35 bp, Artificial Sequence
  Description of Artificial Sequence  Synthetic Primer
aatttgtcgacagaaaatgtagaagcagtaaaagc

SEQ ID NO: 15 - DNA, 32 bp, Artificial Sequence
  Description of Artificial Sequence  Synthetic Primer
gggtggtacctgttcatgttgttaacaactcc

SEQ ID NO: 16 - DNA, 39 bp, Artificial Sequence
  Description of Artificial Sequence  Synthetic Primer
aaattggtaccagaatctaaataattaactaaatatagc

SEQ ID NO: 17 - DNA, 31 bp, Artificial Sequence
  Description of Artificial Sequence  Synthetic Primer
cattggaattcttgctatcgctgtgattgcg

SEQ ID NO: 18 - DNA, 35 bp, Artificial Sequence
  Description of Artificial Sequence  Synthetic Primer
gatccggtacccggattttatgaccgatgatgaag

SEQ ID NO: 19 - DNA, 35 bp, Artificial Sequence
  Description of Artificial Sequence  Synthetic Primer
gatccggtaccttagaaatccctttgagaatgttt

SEQ ID NO: 20 - DNA, 23 bp, Artificial Sequence
  Description of Artificial Sequence  Synthetic Primer
cgtagctggagccaccgcagttc

SEQ ID NO: 21 - DNA, 21 bp, Artificial Sequence
  Description of Artificial Sequence  Synthetic Primer
aaaatgctaccaaaaacttga

(19) United States
(12) Patent Application Publication
(10) Pub. No.: US 2005/0220788 A1
(43) Pub. Date: Oct. 6, 2005

(54) Use of molecules which interact with the haptoglobin receptor ligand binding

(75) Inventors: Eszter Nagy, Vienna (AT); Agnieszka Dryla, Vienna (AT); Dieter Gelbmann, Andau (AT)

Correspondence Address: FULBRIGHT & JAWORSKI L.L.P., 600 CONGRESS AVE., SUITE 2400, AUSTIN, TX, 78701, US

(73) Assignee: Intercell AG, Vienna (AT)

(21) Appl. No.: 10/512,373

(22) PCT Filed: Jun. 18, 2003
(86) PCT No.: PCT/EP03/06438

§ 371 Date: Oct. 25, 2004

(30) Foreign Application Priority Data
Oct. 3, 2002 (AT) A 1497/2002

(51) Int. Cl. A61K 39/395; C07K 16/28; C12Q 1/68; C12N 5/06; C07H 21/04

(52) U.S. Cl. 424/144.1; 530/388.22; 536/23.53; 435/6; 435/334

(57) ABSTRACT

The present invention provides the use of molecules, which interact with the haptoglobin receptor ligand binding for the preparation of a medicament to prevent and treat Staphylococcus infection.

The present invention relates to the preparation of medicaments to prevent and treat Staphylococcus infection.

Staphylococcal infections are imposing an increasing threat in hospitals worldwide. S. aureus is one of the most common sources of nosocomial infections. It causes many different infections, such as asymptomatic nasal carriage, from mild to severe skin and wound infections, peritonitis, osteomyelitis, pneumonia, urinary tract infections. The most severe conditions are bacteremia and sepsis, when this bacterium resides in the blood and ‘travels’ all over the body. The appearance and disease causing capacity of staphylococci are related to the wide-spread use of antibiotics which induced and continue to induce multidrug resistance. For that reason, medical treatment against staphylococcal infections cannot rely only on antibiotics any more. A tactic change in the treatment of these diseases is needed, which aims to prevent infection or interfere with bacterial mechanisms promoting disease (reviewed in Cossley, ed., 1997).

It is therefore an object of the present invention to provide alternative means of treatment, for combating the Staphylococcus infection.

The object is solved by the present invention providing the use of molecules which interact with the haptoglobin receptor ligand binding for preparation of a medicament to prevent and treat Staphylococcus infections.

The haptoglobin receptor, which was earlier referred to as LPXT-Gp5, yet without any function (especially receptor function) connected therewith, was identified as a prominent antigen both by bacterial surface display and by proteomics using human sera from patients suffering from different S. aureus infections (WO 02/059148 A, Etz et al, 2002, Vytvytska, 2002). It belongs to a class of cell surface proteins in S. aureus that are involved in direct interaction with host tissues, host cells and molecules (Patti et al., 1994; Schneewind et al., 1995). The present invention provides new uses connected to the biologic function of this protein, which turned out to act as haptoglobin receptor mediating iron uptake of S. aureus and influencing phagocytic killing.

One of the most growth restrictive factors for S. aureus and other pathogens is iron limitation. With present invention, it is the first time possible to address the iron uptake pathway of S. aureus for the preparation of a medicament for the prevention and treatment of S. aureus infections. Iron as a co-factor for a vast number of enzymes is an essential, growth-limiting nutrient for bacteria. Iron overload results in toxicity, mainly due to uncontrolled redox cycling, enzyme inhibition, the formation of hydroxyl radicals that strongly react with all kinds of bio-molecules, of which DNA damage has the most deleterious consequences. Therefore a fine balance between iron starvation and iron poisoning is critical. Iron is known to play a role in the susceptibility to and outcome of several infections. Iron concentration below and above a critical level is weakening the antimicrobial defence of the body. It has been known anecdotally in the medical field that iron supplementation (especially intravenous) can exacerbate chronic, unapparent diseases, best known example being tuberculosis. This phenomenon is reproduced in several animal models (Lounis et al, 2001).

In vivo, (growing in the host) pathogenic bacteria face iron restriction, since in eukaryotic organisms there is no or minute amount (10⁻¹²⁻¹⁵ M) of free iron, due to the fact that under aerobic condition and physiologic pH, Fe ion is insoluble and toxic. Iron is either complexed by small inorganic substances (such as citrate) or bound to proteins. Extracellular pathogenic bacteria have to extract iron from Fe-binding plasma (extracellular) proteins, such as transferrin, lactoferrin, free (plasma) hemoglobin and hemopexin. Intracellular iron is built into heme and non-heme iron binding proteins, the most abundant of them being hemoglobin (in red blood cells), myoglobin (in muscles) and cytochrome C (in every cells). Excess iron is sequestered intracellularly by a specialised storage protein, ferritin.

Since bacteria are highly dependent on external source of iron, specialised iron acquisition systems are prerequisite for the survival and growth of pathogenic bacteria in their host. Numerous bacterial proteins are involved in microbial iron uptake and transport and considerable variation has been found in the uptake systems used by different bacterial species. There are two main mechanisms employed by bacteria to extract iron from the host iron-binding proteins. There are several surface receptors identified both in Gram− and Gram+ bacteria directly binding to host iron-binding proteins, bringing them to close proximity to the bacterial surface where stripping off the iron or heme from these proteins occurs. This process is followed by specific transport through the membranes by specialised or general-purpose transport proteins (reviewed in Braun, 2001). The other mechanism to acquire iron is to produce and secrete low molecular weight molecules, so called siderophores that have very high affinity for Fe³⁺ and heme. This extremely high affinity (10⁻¹² M) enables these molecules to scavenge iron from protein-bounds. The iron and heme loaded siderophores are taken up through specific receptors, and the iron is utilised intracellularly. Different bacteria prefer one or the other, or even use both methods and express several different receptors for binding to host iron-binding proteins or for importing low molecular weight iron-chelating compounds such as heme, citrate or siderophores. Pathogenic Neisseriae (N. meningitidis, N. gonorrhoe, S. pneumoniae or S. pyogenes) for example, do not possess enzymes and receptors for the production and uptake of siderophore components, and solely rely on scavenging iron through capturing host iron-binding proteins. Homologous transferrin-binding (TbpA, B) and lactoferrin-binding (LbpA,B) proteins have been identified in Neisseriae and Haemophilus influenzae (Cornelissen et al, 1992; Pettersson et al, 1994; Bonnah et al, 1995; Biswas and Sparling, 1995; Gray-Owen et al, 1995; Schryvers, 1988). In addition, Neisseriae possess receptors for binding to hemoglobin (HmbR) and haptoglobin-hemoglobin complexes (HpuA, B) (Lewis et al, 1997; Kahler et al, 2001; Stojiljkovic et al, 1996). Hemoglobin-haptoglobin receptors were also found in Haemophilus influenzae (Jin et al, 1996; Ren et al, 1998). The expression pattern and ligand specificity of these iron-binding proteins are well characterised. It has been determined that they are required for growth in low iron containing media, since knock out strains are growth restricted, but not completely deficient, since the redundant nature of the heme acquisition systems expressed by H. influenzae or Neisseriae. The presence of circulating antibodies in sera of convalescent patients with H. influenzae or N. mengitidinis infections are indicators of in vivo expression.

It was shown in the present invention that recombinant LPXTGp5 can be used in affinity purification to bind and purify the serum haptoglobin from human plasma. Commercially available haptoglobin was also shown to have the ability bind to LPXTGp5 protein, either as recombinant protein or directly isolated from bacterial cells. Therefore, it is clearly shown by the present invention that LPXTG5 functions as a haptoglobin receptor for binding haptoglobin to the S. aureus surface thereby enabling iron uptake for this bacterium.

The most abundant Fe-binding protein is hemoglobin (Hb), which is mainly intracellular (red blood cells). However, due to the physiologic turnover of red blood cells (t_{1/2}=180 days) there is always a low amount (˜10 μg/ml) of free hemoglobin in the serum. During hemolysis, high amount of Hb is released into the plasma. Plasma (free) Hb is immediately complexed to haptoglobin (Hp), an abundant (0.5-2 mg/ml) plasma glycoprotein through an extremely high affinity binding (Kₐ>10⁻¹⁵ M). Massive hemolysis can deplete out the serum haptoglobin, thus low Hp concentration is a diagnostic value for hemolysis. One of the major roles of haptoglobin binding to hemoglobin is to prevent filtration of Hb (64-kDa) into the urine by the kidney glomeruli, simply by increasing the size of protein complexes above the filtration limit (>70 kDa). Hp-Hb complex formation prevents two dangerous conditions to be developed. The immediate danger is the high concentration of Hb in the glomeruli, which plugs the flow of filtrate and prevents excretion of urine, causing acute renal failure. The chronic danger is the loss of iron for the body leading to iron deficiency, mainly anaemia. In order to prevent kidney damage and iron loss Hp-Hb complexes are taken out from the circulation by the RES (ReticuloEndothelial System) of the liver. RES cells are equipped with high affinity receptors, specific for haptoglobin binding to Hb.

It was known that plasma proteins related to iron-metabolism, such as haptoglobin, hemopexin and lactoferrin have immunological functions and contribute to normal infection resistance.

Haptoglobin is an acute phase protein with presumed anti-inflammatory activities. Its hepatic expression is increased by the pro-inflammatory cytokines IL-6 and IL-1 (Baumann et al, 1990). In addition, TNF-alpha seems to promptly increase the level of Hp at sites of infection or injury, leading to the modulation of the acute inflammatory response (Berkova et al, 1999). Recently, it was demonstrated that haptoglobin can also be expressed by lung epithelial cells, and it is likely to contribute to microbial resistance locally (Yang et al, 2000). Moreover, haptoglobin has been implicated in the regulation of phagocytic function. Human phagocytic cells (both polymorphonuclear granulocytes as well as monocytes, but not eosinophils) contain haptoglobin within their specific granules. These haptoglobin stores reflect specific uptake of haptoglobin from the extracellular milieu. Moreover, haptoglobin, like other granule moieties, is exocytosed during phagocytosis (Wagner et al, 1996). In in vitro assays Hp was shown to inhibit phagocytosis and intracellular killing of bacteria by granulocytes (Rossbacher et al, 1999). The assumed function is down-regulation of oxidative burst during phagocytosis in order to prevent oxidative damage of the phagocytes. A novel Hp-Hb receptor (CD163) on macrophages has been described recently (Kristiansen et al, 2001). CD163/Hb scavanger receptor is responsible for the hepatic uptake of Hb-Hp complexes from the circulation. In addition, the neutrophil granulocyte surface integrin CD11b/CD18 has been shown to bind Hp (El Ghmati et al, 1996). These data suggest that haptoglobin has important biological function in host defence against infections and inflammation, acting as a natural antagonist for receptor-ligand activation of the immune system

Interestingly, there are some reports indicating that Hp has even direct inhibitory effect on the growth of bacteria, e.g. on Streptococcus pyogenes (Delanghe el al, 1998b). A haptoglobin homologue, haptoglobin-related protein (HRP) was identified as essential component of a trypanosome lytic complex, responsible for direct killing of T. cruci (Smith et al, 1995).

There is a haptoglobin gene polymorphism in human population, resulting from a duplicated gene portion encoding for the a chain of Hp. Three phenotypes of the antioxidant protein haptoglobin are known: Hp 1-1, Hp 2-1 and Hp 2-2. These different phenotypes are correlated with susceptibility to different diseases, such as bacterial and viral infections (e.g. AIDS), diabetes, cardiac disease, etc (Delanghe et al, 1998a; Hochberg et al, 2002; Van Vlierberghe et al, 2001). It is mainly explained by different molecular sizes (monomer vs. oligomer formation), consequently different tissue penetration and local anti-oxidative activity. Hp is able to counteract oxidative damage caused by Hb-induced Fenton reaction.

Haptoglobin coating of S. aureus may result in reduced oxidative damage of bacteria within the phagosomes due to the well documented anti-oxidative activity of Hp. Alternatively, Hp bound to the surface of S. aureus may provide escape from killing, by modulating the route of entry into professional phagocytes (through haptoglobin receptors), resulting in free bacteria in the cytoplasm instead of in phagocytic granules.

According to the invention, molecules are provided which interact with haptoglobin receptor mediated binding of ligands which in turn will lead to iron starvation of bacteria, especially S. aureus. The “interaction” as used in the present invention relates to any interaction which leads to an impediment of the binding of ligands by this haptoglobin receptor in the pathogen. Preferably, the interaction is performed by disrupting this mechanism, i.e. complete inactivation or blocking of this pathway. However, also a significant reduction of the functionality of the pathway (e.g. by competitive reactions) is often sufficient for combatting the disease caused by the pathogen comprising the haptoglobin receptor.

It could be shown with the present invention that the predicted open reading frame for LPXTGp5 gene (SA1552 in S. aureus strain N315 according to the annotation of Kuroda et al., 2001) (SEQ ID. No 1) encodes for an 895 amino acid long protein with a typical signal peptide sequence at the N-terminus and typical Gram+ anchor motif sequences at the C-terminus, comprising of LPXTG motif, hydrophobic membrane spanning region and positively charged tail (SEQ ID. No 2).

It should be noted that under the present invention, “Haptoglobin Receptor” is the LPXTGp5 protein (SEQ ID. No 2) encoded by the LPXTGp5 gene (SEQ ID. No 1) and is also designated as “HarA”, which stands for Haptoglobin Receptor A, which possesses specific ligand binding activity towards human plasma haptoglobin and haptoglobin-haemoglobin complexes.

According to a preferred embodiment of the present invention, the molecules, which interact with the haptoglobin receptor binding of ligands are selected from the group consisting of haptoglobin receptor antibodies, haptoglobin mimotopes binding to a peptide according to SEQ ID NO 2 (haptoglobin receptor), or a fragment thereof.

Under “haptoglobin receptor antibodies”, any antibody or antibody fragment or derivative is understood which exhibits the binding affinity towards the haptoglobin receptor according to the present invention. These may be polyclonal or monoclonal antibodies, single chain antibodies or other fragments comprising the variable binding domains of the antibody molecule.

An efficient way to inhibit binding to Haptoglobin (Hp) is through specific antibodies raised against LPXTGp5. In addition to neutralising its function as HpR, the same antibodies can support opsonophagocytosis, since Hp-binding region must be surface exposed. There are examples for protective vaccines used in animal models, which are composed of bacterial iron uptake receptor proteins (Webb and Cripps, 1999). Webb and Cripps demonstrated that immunisation with recombinant transferring-binding protein (TbpB) enhances clearance of nontypeable Haemophilus influenzae from lung by stimulating protective responses (antibody production) in a rat model.

Moreover, anti-LPXTGp5 antibodies can be used for the development of effective inhibitors and antagonists, such as mimotopes. These antagonists are preferably peptides or small inorganic or organic molecules. Alternatively, the identification of HpR can lead to small drug inhibitors and be part of antibacterial chemotherapy or chemoprophylaxis.

According to a preferred embodiment of the present invention, the haptoglobin receptor antibodies or haptoglobin mimotopes bind to a polypeptide selected from the group consisting of SEQ ID NO 4 (D1) and SEQ ID NO 6 (D2), or a fragment thereof.

Within the course of the present invention two highly homologous domains (D1 and D2) have been identified which are located in the extracellular part of the LPXTGp5 protein. Both isolated domains are able to bind to haptoglobin purified from human plasma.

According to one aspect, the present invention provides also a gene encoding a haptoglobin binding molecule (i.e. a haptoglobin receptor). The present invention therefore also relates to such genes comprising the sequence according to SEQ.ID.NO.1. Nucleic acids encoding the D1 or D2 domains according to SEQ ID NO. 3 and SEQ ID NO. 5 and fragments thereof encoding haptoglobin binding polypeptides are also nucleic acid molecules according to the present invention.

The nucleic acid sequences according to the present invention may be present e.g. as RNA or DNA; they may also be present together with appropriate promotor-, enhancer-, marker-, etc. sequences e.g. in a vector, such as a plasmid or viral vector, allowing expression of the polypeptide or the mRNA in a target cell, tissue or body fluid. The Fur box according to SEQ.ID.NO.7 is a preferred regulatory element to be used according to the present invention. The present nucleic acids also encompass nucleic acid sequences which encode haptoglobin receptors or haptoglobin binding fragments thereof, the nucleic acids stringent-ly hybridising to SEQ.ID.Nos 1, 3, 5 and 7, respectively (or their complementary sequences). Possible stringent condition for hybridising are e.g. 6×SSC (as defined in e.g. Sambrook et al 1989, Molecular cloning; A Laboratory Approach).

According to another aspect, the present invention provides isolated polypeptides comprising a polypeptide selected from the group consisting of SEQ ID NO 4, SEQ ID NO 6 and haptoglobin binding fragments thereof as well as homologous domains. Under “homologous domains” all haptoglobin binding domains are understood which comprise a sequence homology to SEQ ID NOs 4 or 6 of at least 40%, preferably at least 70%, especially at least 90%, as calculated by the SIM (Expasy) program (secondary structure determination (e.g. for domain definition; also for homology) may be done e.g. by PSIPRED Prediction Alignment Program).

Significant homology exists only among domains and neighbouring region:

| | LPXTGp5D1 | LPXTGp5D2 |
|---|---|---|
| LPXTGp5D2 | 142 aa | 142 aa |
| | 54.9% | 100% |
| LPXTGp6D | 147 aa | 194 aa |
| | 45.6% | 67.5% |
| LPXTGp7 | 45 aa | 86 aa |
| | 31.1% | 24.4% |

According to yet another aspect of the present invention a synthetic conjugate comprising a peptide according to SEQ ID NO 4 linked by a non-naturally occurring linker to a peptide according to SEQ ID NO 6 is provided. In principle any conjugate which does not interfere with Hp binding is useable for the present invention, e.g. GST (Gluthatione-S-transferase, His-tag, FLAG-tag, etc.).

According to a preferred embodiment of the present invention the non-naturally occurring linker is a polypeptide.

According to another aspect of the present invention the present invention is provided with the use of antisense technology. Therefore, the haptoglobin receptor expression in the pathogen is blocked or largely inhibited by antisense nucleic acid molecules which bind to the haptoglobin receptor mRNA or its regulatory elements. The molecules used for interacting with haptoglobin receptor ligand binding is therefore an anti-sense nucleic acid binding to the haptoglobin receptor gene or to a regulatory element for the expression of the haptoglobin receptor gene, especially the Fur box according to SEQ ID NO 7.

According to a further aspect of the present invention there is provided a nucleic acid hybridising under a stringent condition to a nucleic acid sequence selected from the group consisting of SEQ ID NO 7.

Stringent hybridisation conditions are well known in the art (see above). Optimal hybridisation conditions can be calculated if the sequences of the nucleic acid is known. For example, hybridisation conditions can be determined by the GC content of the nucleic acid subject to hybridisation (Sambrook et al 1989, Molecular cloning; A Laboratory Approach).

In prokaryotes, iron metabolism is mainly regulated at the level of gene transcription. During evolution highly regulated, complex and redundant uptake systems have developed and expression of a large number of genes (>40 in some cases) is directly controlled by the prevailing intracellular concentration of Fe²⁺ via its complexing to regulatory proteins. The best characterised and most conserved among almost all bacteria is the Fur repressor (ferric uptake regulator). Fur directly senses changes in the intracellular iron concentration being an iron binding protein. At sufficient or high concentration iron is bound to Fur. Iron-binding enables the protein to bind to certain DNA sequences, called fur boxes, repressing transcription of target genes. Under iron limiting conditions Fe²⁺ easily dissociates from Fur-Fe complexes allowing Fur-regulated genes to be transcribed (Escolar et al, 1999). Importantly, expression of virulence factors is coupled to iron starvation (e.g. Shigella toxin, colicins, hemolysins), suggesting that low iron concentration is a global signal for pathogenic bacteria that they are “on the battle fields”, that is inside the body. It can be teleologically justified since cytotoxins, hemolysins result in the release of iron binding proteins, such as hemoglobin and myoglobin, which are excellent sources of iron for bacterial growth.

Relatively little is known about iron transport and regulation in Gram+ bacteria in general, and in staphylococci, in particular. Staphylococcus aureus genome encodes three ferric uptake regulator (Fur) homologues: Fur, PerR, and Zur. PerR was found to control transcription of the genes encoding the oxidative stress resistance proteins catalase (KatA), alkyl hydroperoxide reductase (AhpCF), bacterioferritin comigratory protein (Bcp), and thioredoxin reductase (TrxB). Furthermore, PerR regulates transcription of the genes encoding the iron storage proteins—ferritin and the ferritin-like Dps homologue, MrgA (reviewed in Horsburgh et al, 2001). Moreover, it was shown that S. aureus can utilise several hydroxamate siderophores for growth under iron-restricted conditions (Sebulsky et al, 2000). The sir (siderophore regulation) operon has been proposed to constitute a siderophore transport system in S. aureus. It possesses receptors and cytoplasmic membrane-associated traffic ATPases that are involved in the specific transport of iron(III)-hydroxamate complexes (Sebulsky et al, 2001). However, little is known about acquiring iron through direct binding to host iron binding proteins. Surface GAPDH, a 42-kDa S. aureus protein was implicated as being the transferrin receptor (Modun and Williams, 1999). Very recently, an LPXTG protein has been identified as the transferrin receptor for S. aureus (Taylor and Heinrichs, 2002). The present invention showed that the nucleotide sequences upstream of LPXTGp5 correspond to consensus fur binding box between −53 and −35 bps upstream from the starting ATG codon. It was shown in the present invention that LPXTGp5 was constitutively expressed in fur deletion mutant S. aureus strain on one hand, on the other hand, the expression was iron-regulated in wild type S. aureus strains where fur gene was intact.

According to another aspect, the present invention provides a process for isolating molecules, which interact with haptoglobin receptor ligand binding, characterised by the following steps:
providing haptoglobin receptor polypeptides or haptoglobin binding fragments thereof on a solid surface,
binding labelled haptoglobin to said immobilised haptoglobin receptor polypeptides or haptoglobin binding fragments thereof to form a complex between immobilised haptoglobin receptor polypeptides or haptoglobin binding fragments thereof and labelled haptoglobin,
contacting said complex with a pool containing candidate molecules,
determining those molecules of said pool, which replace said labelled haptoglobin in said complex, and
isolating said molecules replacing said labelled haptoglobin in said complex.

With this process it is possible to isolate molecules competing with haptoglobin for the haptoglobin binding site of the haptoglobin receptor.

An equivalent process according to the present invention for isolating molecules, which interact with haptoglobin receptor ligand binding, is characterised by the following steps:
providing haptoglobin immobilised on a solid surface,
binding labelled haptoglobin receptor polypeptides or hapto globin binding fragments thereof to said immobilised hapto globin to form a complex between immobilised haptoglobin and labelled haptoglobin receptor polypeptides or haptogolobin-binding fragments thereof,
contacting said complex with a pool containing candidate molecules,
determining those molecules of said pool, which replace said labelled haptoglobin receptor polypeptides or haptoglobin-binding fragments thereof in said complex, and bind to immo. bilised haptoglobin,
isolating said molecules of said pool bound to immobilised haptoglobin.

With this process it is possible to isolate haptoglobin receptor mimotopes.

A further equivalent process according to the present invention for isolating molecules, which interact with haptoglobin receptor ligand binding, is characterised by the following steps:
providing a pool of candidate molecules,
removing and isolating from said pool those molecules which bind to immobilised haptoglobin receptor or haptoglobin binding fragments thereof,
removing and isolating from said pool those molecules which bind to immobilised haptoglobin,
contacting the remaining pool of candidate molecules with an immobilised complex formed between haptoglobins and hapto globin receptors or haptoglobin binding fragments thereof, and
isolating said molecules which bind to said immobilised complex.

With this process, it is possible to isolate molecules which specifically bind to the haptoglobin/haptoglobin receptor complex and not to the (non-complexed) single components. Since complexes are in vivo present only on the pathogen cell surface, it is possible to combine these complex-specific molecules with appropriate pathogen-combatting molecules, e.g. specific antibiotics, to achieve a site directed control of the pathogen.

In a preferred embodiment of the invention said haptoglobin binding fragment is selected from the group consisting of SEQ ID No.4, SEQ ID No.6, and fragments thereof or combinations of these fragments.

As shown in the examples of the present invention, an in vitro, Elisa based assay and an in vitro, FACS based assay can be established for measuring the competitive binding of LPXTGp5 or a fragment thereof, e.g. D1 and D2, to haptoglobin. This type of assay systems is very useful for screening, isolating molecules that interact or disrupt LPXTGp5 and haptoglobin interaction.

In a preferred embodiment of the invention said haptoglobin receptor is S. aureus haptoglobin receptor.

In a still preferred embodiment of the invention said haptoglobin is a mammalian, especially a human haptoglobin.

The invention is described in more detail by the following examples and figures, but it is not limited thereto.

### FIGURES

FIG. 1 shows structure of LPXTGp5 protein and comparison of secondary structure between LPXTGp5 and other staphylococcal proteins having homologous domains.

FIG. 2 shows recombinant LPXTGp5 by gel electrophoresis, protein staining and immunoblotting.

FIG. 3-4 show IgG levels against rLPXTGp5, D1 and D2 measured in sera of patients suffering from different S. aureus infections and of healthy donors

FIG. 5 shows binding of human plasma proteins to recombinant LPXTGp5.

FIG. 6 shows haptoglobin binding to native LPXTGp5 expressed in in vitro grown S. aureus cells.

FIG. 7 shows growth condition dependent LPXTGp5 expression in S. aureus.

FIG. 8 shows alignment of fur box sequences and iron and Fur regulated expression of LPXTGp5.

FIG. 9 shows haptoglobin binding by rLPXTGp5 domains in an ELISA based assay.

FIG. 10 shows haptoglobin binding to live S. aureus cells measured in a FACS based assay.

FIG. 11 shows inhibition of haptoglobin binding to S. aureus in a presence of D1 domain.

FIG. 12 shows haptoglobin-binding to S. aureus 8325-4 and LXTG-p5KO.

FIG. 13 shows S. aureus growth enhancement by Hp-Hb complexes.

FIG. 14 shows haptoglobin Receptor binds to haptoglobin-haemoglobin complexes.

### EXAMPLES

### Methods and Experimental Procedures

Bacterial Strains and Culture Conditions

Staphylococcus aureus wild-type strain 8325-4 (Novick, 1967), clinical isolate COL (Shafer and landolo, 1979) and restriction-deficient strain RN4220 (Kreiswirth et al., 1983) were from our laboratory's strain collection. Staphylococcus aureus fur mutant (Horsburgh et al., 2001) was a kind gift from Simon Foster (Sheffield University, UK). Staphylococcus aureus strains were cultured in BHI (brain heart infusion) broth or RPMI 1640 tissue culture medium (with 25 mM Hepes buffer and L-Glutamine, Gibco BRL), used as a poor growth medium low in iron. Iron supplementation was achieved by the addition of FeCl₃ to the RPMI medium to a final concentration of 25 μM. Commercially available E. coli strains BL21 and ElectroMAX DH10B (Invitrogen) used for recombinant protein expression and for cloning purposes, respectively, were grown in Luria-Bertani broth (LB). When included, antibiotics were added at the following concentrations: for E. coli ampicillin, 100 μg ml⁻¹; erythromycin, 300 μg ml⁻¹; for S. aureus erythromycin, 5 μg ml⁻¹; lincomycin, 25 μg ml⁻¹; and tetracycline, 5 μg ml⁻¹. Unless otherwise stated, all bacterial growth was carried out at 37° C. with shaking at 150 r.p.m.

Bacterial Lysate Preparation

Total bacterial lysate was prepared with lysostaphin digestion (100 μg ml⁻¹ in PBS) for 30 min at 37° C. in the presence of protease inhibitors (CompleteΣ, EDTA-free tablets, Roche). In addition to enzymatic digestion, cells were disrupted by sonication using a microsonicator (Bandelin Sonopuls, HD 2200, Germany). After centrifugation the soluble fraction was recovered and protein concentration was determined by the Bredford method (Bio-Rad Protein Assay).

Expression of Recombinant HarA

The cDNA encoding for HarA was amplified from S. aureus COL genomic DNA by gene specific oligonucleotides HARA1 and HARA2 with incorporated BsaI sites (Table 1). Restriction enzyme digested PCR product was cloned into the BsaI cleaved pASK-IBA4 vector downstream of a sequence, which codes for the Strep-tag II (IBA, Göttingen). The resulting gene lacked sequences corresponding to the signal peptide (QAQA, AENT) and the C-terminal part, downstream from the sortase cleavage site (LPKT, G). The recombinant protein was purified from bacterial extracts of anhydrotetracyclin induced BL21 E. coli through StrepTactin affinity chromatography (according to the manufacturer's instructions). In addition to the full-length protein, two truncated versions of HarA were also generated by amplifying DNA sequences corresponding to the predicted D1 and D2 domains. Polymerase chain reaction products were generated using oligonucleotide primers MOL1031 and MOL1032 or MOL1033 and MOL1034, respectively (Table 1) then digested with BamHI-SalI for insertion into BamHI-SalI digested pGEX-4T-3 (Amersham Biosciences). GST-fusion proteins were extracted from IPTG induced BL21 E. coli cells by sonication (in buffer: 50 mM Tris-HCl pH 8.0, 100 mM NaCl, 1 mM EDTA), and purified on a Glutathione Sepharose 4B affinity column (Amersham Biosciences) from soluble bacterial fractions. Recombinant proteins were eluted either by thrombin digestion (50 U ml-1, for 3 h at RT), or with 10 mM glutathione.

TABLE 1
List of primers.

| | |
|---|---|
| Name | Nucleotide sequence |

| | |
|---|---|
| HARA1 | 5-AGGCATGGTCTCTGCGCCGCAGAAAATACAAATACT-3 |
| HARA2 | 5-ATTGCTGGTCTCTTATCAAGTTTTTGGTAGCATTTT-3 |
| MOL1031 | 5-AATGGATCCGCGGATGAATCACTTAAAGATGC-3 |
| MOL1032 | 5-TATGTCGACCTAAAGTGAAGGATCGTTATAAATAGG-3 |
| MOL1033 | 5-AATGGATCCGCAGATGAATCACTACAAGATGC-3 |
| MOL1034 | 5-TATGTCGACCTAGTCGTCTGGGTTATTAGTAATAGG-3 |
| MOL1313 | 5-AATTTGTCGACAGAAAATGTAGAAGCAGTAAAAGC-3 |
| MOL1314 | 5-GGGTGGTACCTGTTCATGTTGTTAACAACTCC-3 |
| MOL1315 | 5-AAATTGGTACCAGAATCTAAATAATTAAC |
| | TAAATATAGC-3 |
| MOL1316 | CATTGGAATTCTTGCTATCGCTGTGATTGCG-3 |
| MOL1317 | 5-GATCCGGTACCCGGATTTTATGACCGATGATGAAG-3 |
| 317 | |
| MOL1318 | 5-GATCCGGTACCTTAGAAATCCCTTTGAGAATGTTT-3 |

Restriction sites underlined

Affinity Purification of Human Plasma Proteins

First, human plasma was depleted of IgG by binding to UltraLink Immobilized Protein G beads (PIERCE). Briefly, 1 ml of plasma diluted 1:2 in PBS (pH 7.4) was applied twice on a Protein G Sepharose column and the flow-through collected. IgG depleted plasma (˜60 mg total protein) was incubated with 20 μg of StrepII-tagged recombinant protein immobilized on StrepTactin agarose (IBA, Göttingen). Following extensive washing of the beads in PBS, proteins were eluted with 100 μl of isoelectric focusing sample buffer (IEF: 10 M urea, 4% CHAPS, 0.5% SDS, 100 mM DTT).

Affinity Purification of S. aureus Proteins with Purified Haptoglobin

The haptoglobin affinity column was prepared by binding 200 μg biotinylated haptoglobin (Hp:biotin=1:10) to 40 μl of Streptavidin Gel Ultralink Plus (PIERCE). Two milligrams of total protein extracted from S. aureus 8325-4 cells grown in RPMI until the stationary phase was applied to the Hp-column. After extensive washing bound proteins were eluted with 100 μl IEF buffer, and 40 μl of the eluate was analysed by SDS-PAGE followed by immunoblotting.

Two-Dimensional Gel Electrophoresis

High resolution two-dimensional gel electrophoresis was carried out as described elsewhere (Hochstrasser et al., 1988), using the mini-Protean electrophoresis system (Bio-Rad). For the analysis of IgG depleted plasma, 1 μl of sample was diluted up to 10 μl with IEF sample buffer. Elution fractions in sample buffer were loaded directly on the gel. One-dimensional isoelectric focusing was performed at 2625 V-h in 1 mm×10 cm tube gels in a stepwise fashion (10 min at 500 V, 3.5 h at 750 V), using 4% acrylamide (Gerbu, Gaiberg, Germany)/0.1% PDA, 0.035% Nonidet P-40 and 2% ampholytes (pH 3.5-10:pH 4-8:pH 5-7=1:1:2; Merck, Darmstadt, Germany) with degassed 20 mM NaOH as catholyte and 6 mM H₃PO₄ as anolyte. The tube gels were placed on top of 1.0 mm 12% SDS-PAGE slab gels. After 3 min equilibration with 3% SDS, 70 mM Tris base, 0.001% bromphenol blue, the second dimension was run at 15° C. using 0.1% SDS, 25 mM Tris base and 200 mM glycin as electrode buffer. Gels were Coomassie Blue stained. Proteins detectable in this system range: 10-220-kDa, pI 3.5-7.5.

Generation of Anti-HarA Antibodies

Human anti-HarA IgGs were isolated from plasma of a healthy donor determined to have high antibody levels against rHarA in ELISA. Fifty millilitres of plasma was diluted 1:2 in an Immunopure IgG Binding Buffer (PIERCE) and applied to UltraLink Immobilized Protein G beads (PIERCE). IgGs bound to the column were eluted with an ImmunoPure IgG Elution Buffer (PIERCE) and neutralized with 1 M Tris-HCl pH 8.0. Elution fractions were pooled and dialysed against PBS overnight at 4° C. 150 mg of IgGs were incubated with 40 mg of biotin-labelled HarA immobilized on 50 μl of UltraLink Plus Immobilized Streptavidin Gel (PIERCE). After extensive washing, the fractions were eluted with the ImmunoPure IgG Elution Buffer. This purification yielded ˜20 μg IgG, which was tested for specificity in ELISA and immunoblotting with rHarA and several unrelated S. aureus recombinant proteins, as negative controls. Hyperimmune polyclonal immune sera were generated by immunizing rabbits with recombinant proteins representing either the full-length HarA or truncated versions consisting of single domains—D1 and D2. New Zealand White rabbits were immunized three times in 3-week intervals with 250 μg of protein per injection per rabbit before bleeding. Efficient immunization and the presence of specific antibodies were confirmed by ELISA and immunoblotting with the respective recombinant proteins.

Immunoblotting

Proteins were separated by one- or two-dimensional SDSPAGE using a mini-Protean electrophoresis system (Bio-Rad) and transferred to a nitrocellulose membrane (ECL, Amersham Biosciences) using a semi-dry transfer system (Bio-Rad) and visualized by Ponceau S staining. After overnight blocking in 5% milk, purified human anti-HarA IgGs at 100 ng ml⁻¹ concentration or rabbit preimmune or immune sera at 1:10000 dilutions were added, and HRP-labelled goat anti-human IgG (Southern Biotech) or HRP-labelled goat anti-rabbit IgG (Amersham Biosciences) were used for specific detection of the HarA protein. The signal was developed using an ECL detection system (Amersham Biosciences).

Cell Surface Staining with Anti-HarA Antibodies

5×10⁶ S. aureus cells grown in the RPMI medium in the absence or presence of 25 μM FeCl₃, as an iron source, until the late logarithmic phase (OD₆₀₀-0.8-1.0; maximum OD₆₀₀ in RPMI was ˜1.6) were used for staining with polyclonal rabbit anti-HarA antiserum. Non-specific binding of antibodies was prevented by incubation with a human IgG Fc fragment (Jackson ImmunoResearch) at 10 μg sample⁻¹ before the addition of rabbit hyperimmune serum at 1:500 dilution. After washing with PBS, secondary reagent—FITC-labelled anti-rabbit IgG/Fab fragment specific (Jackson ImmunoResearch) was added. All steps were performed on ice, each for 30 min. Finally, cells were washed with PBS, fixed with 2% PFA and fluorescence was quantified by FACScan (Becton Dickinson).

Haptoglobin-Binding Assays

The in vitro ELISA based assays were performed in two different set-ups. First, we used haptoglobin purified from pooled human plasma (SIGMA and FLUKA) as a coating reagent at 10 μg ml⁻¹ concentration in coating buffer (0.1 M Na-carbonate, pH 9.3) and GST-D1 and GST-D2 as binding partners at amounts between 2.5 and 12 pmoles (2-10 μg ml⁻¹). Interactions between Hp and D1 or Hp and D2 were detected with biotin-labelled goat anti-GST mAbs (Abcam, UK) diluted 1:5000 and Streptavidin-HRP (Roche) at 1:10000 dilution. Second, rHarA, D1 and D2 domain proteins were coated in the coating buffer at 10 μg ml⁻¹ concentration and the ligands haptoglobin, haptoglobin-haemoglobin complexes or haemoglobin were added at amounts between 0.08 and 4.0 pmoles. Haptoglobin-haemoglobin complexes were prepared by gentle mixing haptoglobin with haemoglobin (Sigma) at 1:1 molar ratio for 45 min at RT. Complex formation was visualized by CBB staining of native PAGE gels. Binding of the ligand proteins were detected by anti-human haptoglobin (SIGMA) and anti-human haemoglobin (Abcam, UK) monoclonal antibodies at 1:2.000 dilution and HRP-labelled anti-mouse IgG as secondary reagent. Antigen-antibody complexes were quantified by measuring the conversion of the substrate (ABTS) to coloured product based on OD₄₀₅ₙₘ readings in an automated ELISA reader (Wallace Victor 1420). The FACS based assay was performed using purified haptoglobin labelled with biotin (EZ-Link Sulfo-NHS-LCBiotin, PIERCE) at a 10:1 biotin to haptoglobin ratio for 30 min at RT. After removal of free biotin on Nanosep 10K centrifugal devices (Pall, Life Sciences, USA), labelling of haptoglobin was confirmed by immunoblotting using Streptavidin-HRP as a detection reagent. Staphylococcus aureus cells were grown in the RPMI medium in the absence or presence of 25 μM FeCl₃, as an iron source, until the late logarithmic phase (OD600=0.8-1.0). Biotinylated haptoglobin (from 5 to 30 μg) was added to 5×106 cells and incubated for 30 min at RT. After washing with PBS, Streptavidin-FITC (DAKO) at 1:100 dilution was added, then cells were fixed with 2% PFA. Surface binding of haptoglobin was quantified by measuring fluorescence intensity by FACScan (Becton Dickinson).

Iron Dependent Growth

For growth studies iron depleted RPMI medium was used. Iron depletion of the RPMI medium was achieved by batch incubation with Chelex100 (Sigma). Briefly, 10 g Chelex100 was added to 1 L medium and stirred for 4 h at RT. Then the medium was supplemented with divalent ions to 10 μM of CaCl₂ and 100 μM of MgSO₄. Staphylococcus aureus 8325-4 and harA mutant cells were inoculated from a BHI plate and incubated overnight in the RPMI complete medium. Cells were collected, washed and resuspended in iron depleted RPMI medium to reach an OD₆₀₀ of 0.05. Following a 3 h of iron starvation, cells were collected and diluted to OD₆₀₀ of 0.02 in iron depleted RPMI supplemented with various iron sources. The following iron sources were used: ferric chloride at concentration of 25 mM, Hb at 0.5 mM, and Hp-Hb (2:1) complexes at 1 μM and 0.5 μM concentrations. Bacterial growth was monitored by measuring optical density at 600 nm with a Hitachi U-2001 spectrophotometer.

Construction of harA Insertionally Inactivated Mutant

The plasmid for insertional inactivation of harA was constructed using the pAUL-A vector (kind gift of Simon Foster) described before (Chakraborty et al., 1992). 5′ and 3′ flanking regions of the harA open reading frame were generated by PCR using gene specific primers MOL1313, MOL1314 and MOL1315, MOL1316, with added SalI/KpnI and KpnI/EcoRI restriction sites respectively (Table 1). The 1 kb fragments were cloned into the SalI-EcoRI digested pAUL-A vector resulting in the plasmid pAUL-AD. The tetracycline resistance cassette was amplified from pDG1513 (Guerout-Fleury et al., 1995; kind gift of Simon Foster) using primers MOL1317 and MOL1318 with incorporated KpnI restriction sites (Table 1). The KpnI digested PCR fragment containing the 1.5 kb tetracycline resistance cassette (Tc) was then cloned into pAUL-AD. Fifty micrograms of the resulting pAD02 plasmid was transformed into S. aureus RN4220 restriction-deficient transformation recipient by electroporation. Erythromycinresistant transformants were identified at the permissive temperature for plasmid replication (30° C.). Single crossover Campbell-type chromosomal insertions were selected by shifting temperature to 42° C. while selecting on tetracycline. Integration of the pAD02 plasmid into chromosomal DNA was confirmed by PCR analysis. Integration of the tetracycline resistance marker into S. aureus 8325-4 chromosome was achieved by transduction with phage 11. Transduced colonies were further selected for loss of erythromycin and lincomycin resistance, and tested for the lack of the harA gene by PCR using gene specific primers, as well as with Southern blotting.

### Results

### Example 1

### LPXTGp5 is a Highly Immunogenic Novel Cell Wall Protein Expressed In Vivo During Different S. aureus Infections

1/A. Identification of LPXTGp5 as Antigen

Specific anti-bacterial antibodies are molecular proofs of in vivo expression of the corresponding antigens. Identification of antigen-specific serum antibodies is widely used in serodiagnosis of certain pathogens, especially of the non-cultivable ones.

LPXTGp5 was identified as a prominent antigen both by bacterial surface display and by proteomics using human sera from patients suffering from different S. aureus infections (see WO 02/059148 A, Etz et al., 2002, Vytvytska et al, 2002). Five different B-cell epitope regions of the protein were identified by surface display, all being localised to the N-terminus. Based on these data LPXTGp5 is expressed during human S. aureus infections, and widely immunogenic with multiple epitopes in many patients. Bioinformatic analysis identified a novel protein without known function.

1/B. LPXTGp5 Gene and Protein

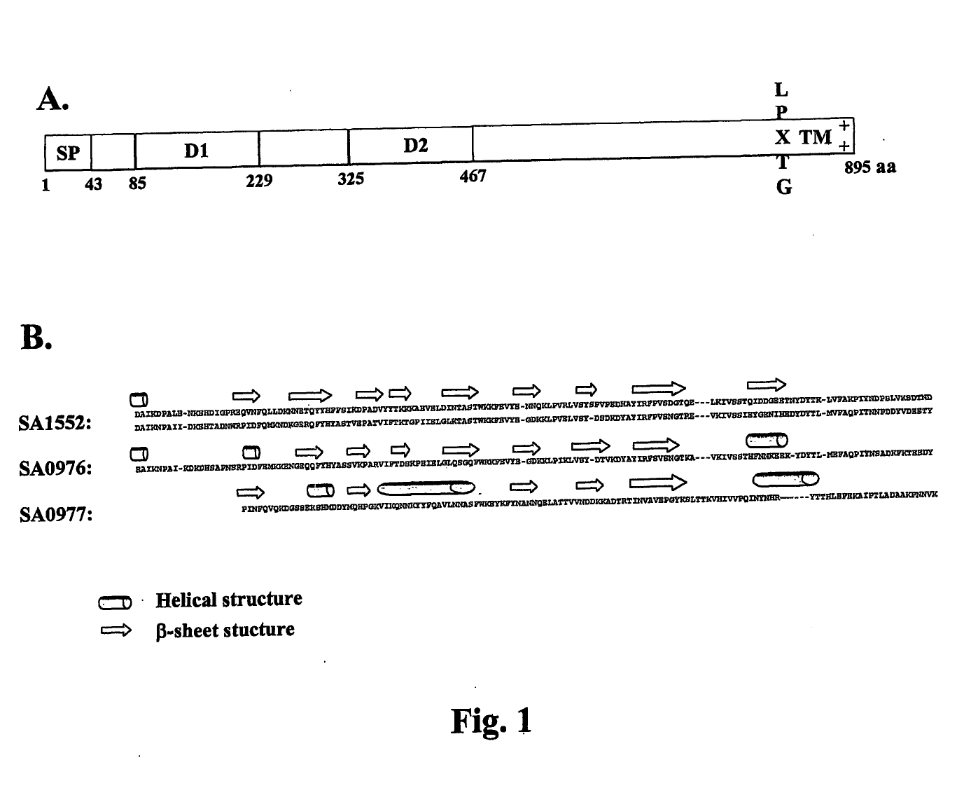

The predicted open reading frame for LPXTGp5 gene is located between 1824064 and 1821380 bps of the S. aureus COL strain according to TIGR annotation (SA1781, InterCell ORF01361; Kuroda et al., 2001) (SEQ.ID.No1). The predicted ORF encodes for an 895 amino acid long protein with a typical signal peptide sequence at the N-terminus and typical Gram+ anchor motif sequences at the C-terminus, comprising of LPXTG motif, hydrophobic membrane spanning region and positively charged tail (SEQ.ID.No2). Both the primary amino acid sequence and predicted secondary structure analysis suggest for the presence of two homologous domains (D1 and D2) with all beta-sheet structures for domains, which are separated by helical or coil regions (FIG. 1). The two domains have ˜145 amino acid residues and show 52% identity and 71% similarity.

Interestingly, sequence homology searches identified similar single domains in three other S. aureus proteins, we named LPXT-Gp6, LPXTGp7, and p7. Remarkably, these three proteins are immediate neighbour genes on the S. aureus chromosomes. LPXTGp7 and p7 seems to be transcribed as one mRNA. moreover, the p7 gene is followed by three predicted membrane proteins, which show homology to ferric ABC transport family of proteins. All four proteins, including p5 are highly conserved among the five S. aureus strains for which genomic information is available. Interestingly, all four proteins were found to be immunogenic with human sera (see e.g. WO02/059148 A). Similarly to LPXTGp5, p6 and p7/p7-like contain fur box sequences, and in very recent publication these proteins were shown to be iron regulated (Mazmanian et al, 2002). The predicted structure of the homologous domains is very similar (PhD) in spite of a moderate amino acid identity of ˜40%. In addition, there are proteins with homologous domains in other Gram+ bacteria, all belonging to the genus Clostridium. Listeria monocyotgenes has a protein, called p64, which has three of this domain. Proteomic analysis suggested that the expression of p64 is iron regulated (Borezee et al, 2000). Bacillus halodurans genome possesses a predicted open reading frame having this domain.

1/C. Generating Recombinant Proteins

cDNA encoding for LPXTGp5 was amplified from S. aureus COL strain genomic DNA by gene specific oligonucleotides 5′-CGTAGCTGGAGCCACCGCAGTTC-3′ and 5′-AAAATGCTACCAAAAACTTGA-3′, respectively. Restriction enzyme digested PCR product was cloned into the BamHI-SalI site of the pASK-IBA4 vector downstream of sequences coding for the Strep-tag II (IBA, Göttingen). The resulting gene lacked sequences corresponding to the signal peptide and the C-terminal end, downstream from the sortase cleavage site (LPXTG). The recombinant protein was purified from bacterial extracts of ampicylin induced BL21 E. coli through Streptactin affinity chromatography (according to the manufacturer's instructions). Although the predicted molecular weight of the 895 aa protein is 101-kDa, the recombinant full-length LPXTGp5 migrated as an ˜130-kDa protein. It is common for bacterial cell wall proteins to migrate slower than their actual size.

In addition to the full-length protein, two different truncated versions of LPXTGp5 were also generated by amplifying DNA sequences corresponding to the predicted D1 and D2 domains, and inserted into BamHI-SalI digested pGEX4T-3. The GST-fusion proteins were extracted from IPTG induced DH10B E. coli cells by lysozyme digestion (in buffer: 50 mM Tris pH 8.0, 100 mM NaCl, 1 mM EDTA), and purified on gluthatione affinity column from soluble bacterial fractions. Recombinant proteins were eluted either by thrombin digestion, or with 10 mM glutathione. Thus, the resulting 145 aa long D1 and D2 recombinant truncated versions were available with or without the GST tag.

1/D. LPXTGp5 is Widely Immunogenic in Humans

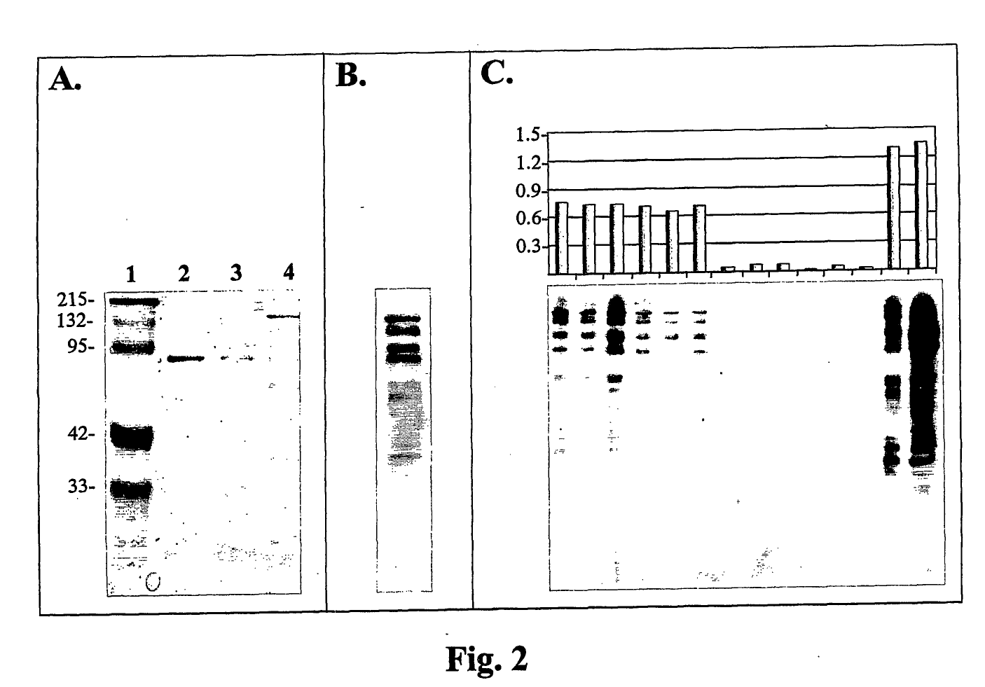

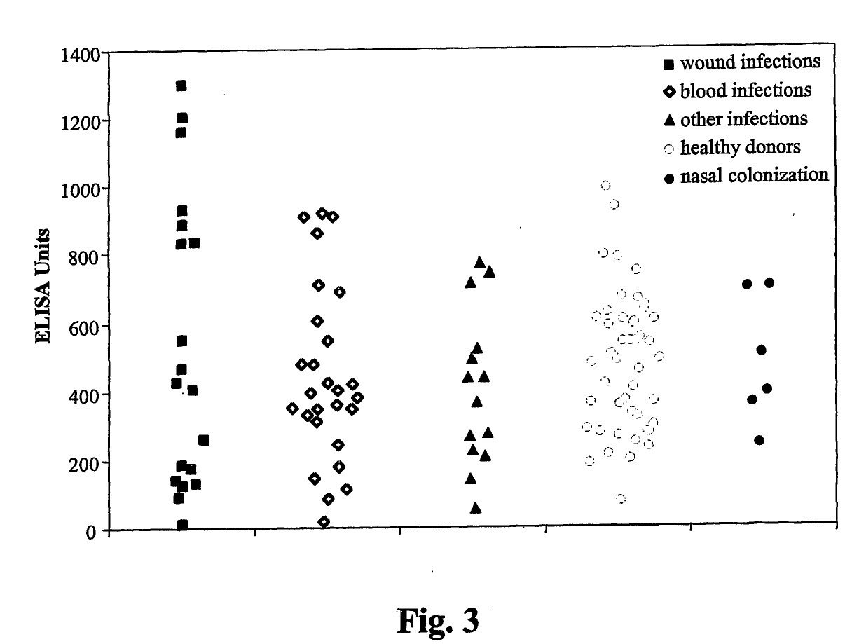

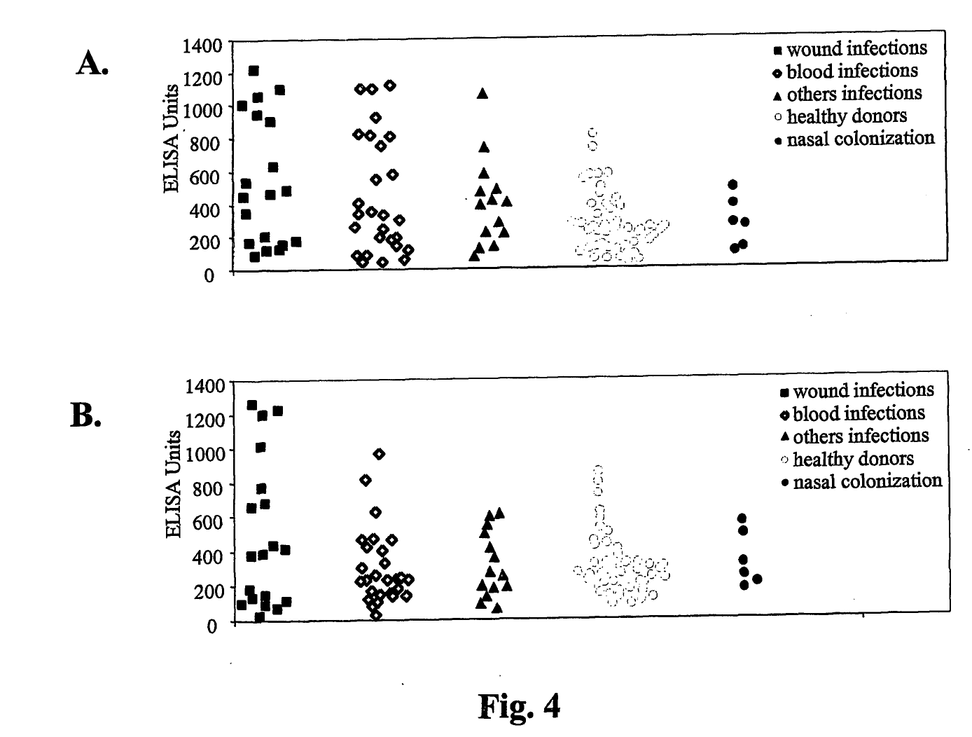

In a series of immunoblot and ELISA experiments, it was determined that human sera, both from patients suffering from different S. aureus infections as well as from healthy individuals contained antibodies against LPXTGp5 (FIG. 2). There were differences in the levels of anti-LPXTGp5 IgG (FIG. 3) and IgA (data not shown) among the different sera, patients with S. aureus wound infections displaying the highest levels. ELISA with D1 and D2 proteins demonstrated that these domains were also highly immunogenic (FIG. 4).

### Example 2

### Identification of Serum Haptoglobin as Binding Ligand for LPXTGp5

2/A. Affinity Purification of Human Plasma Proteins with Recombinant LPXTGP5

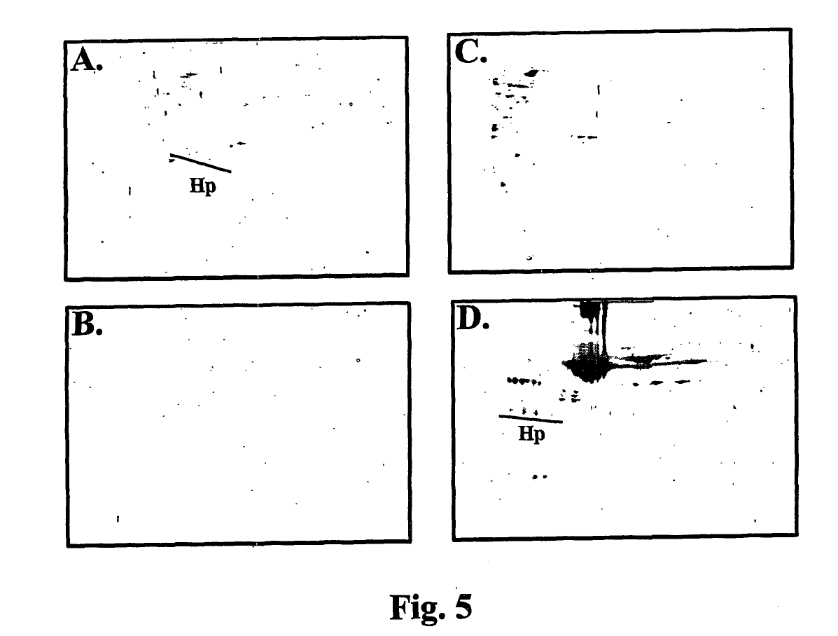

In order to identify binding partner(s) and function for LPXTG-p5, the StrepII-tagged recombinant protein was immobilised on Streptactin agarose (IBA) and IgG-depleted human plasma was applied. Selection of the plasma sample was based on low IgG and IgA titers against rLPXTGp5 by ELISA to avoid undesired immune interactions. Elution fractions of human plasma proteins bound to the LPXTGp5 column, as well as that of a control column of only Streptactin were subjected to 2D-PAGE analysis. Coomassie Blue staining of the 2D gels revealed a group of protein spots with characteristic appearance in the 40- to 45-kDa and pI 4.5-5.5 range (FIG. 5A). These spots were missing from the eluate of the control column (FIG. 5B) and also from the gel of rLPXTGp5 alone (FIG. 5C). Separation of the non-fractionated plasma sample revealed the same group of spots (FIG. 5D) and helped the identification of the corresponding proteins. Based on the characteristics of the protein spots in the 2D gels, with the help of proteomics information available from the Swiss-2DPAGE database (http://us.expasy.org/ch2d/), the 40-45-kDa purified proteins were identified as the subunit of the human plasma/serum glycoprotein, haptoglobin. The characteristic beads-on-a-string appearance is due to the N-linked glycosylation at four potential glycosylations sites at Asn residues described earlier. To confirm this finding, elution fraction from the LPXTGp5 affinity column was subjected to 2D immunoblot analysis using anti-human haptoglobin antibody, and purified human serum haptoglobin was used as positive control. The characteristic 5 spot appearance of the signal in the identical region of the 2D gel was reassuring.

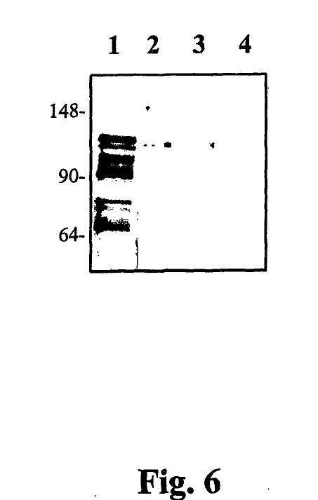

In order to provide unambiguous evidence, commercially available haptoglobin purified from pooled human plasma (cat# 51325, Fluka) was purchased and tested for its ability to bind to LPXT-Gp5 affinity column. Similarly to the experiment using plasma, purified Hp was retained on the column through the interaction with LPXTGp5, since eluates from control Streptactin agarose did not contain haptoglobin. The reverse experiment was also performed, using purified haptoglobin immobilised on Streptavidin agarose beads through biotin labelling and total lysates prepared from S. aureus 8325-4 spa-strain grown to exponential phase in iron depleted (with Chelex 100) RPMI medium. Immunoblot analysis of eluates from haptoglobin coupled beads provided further proof that native LPXTGp5, directly isolated from bacterial cells is indeed a binding partner for this extracellular host glycoprotein (FIG. 6).

2/B. Localisation of Ligand Binding to the D1 and D2 Domains of LPXTGP5

Production of recombinant LPXTGp5 in E. coli always resulted in degradation product (FIG. 2), which is likely due to the big size of the protein and partial incompatibility of expression of this Gram+ cell wall protein in the Gram− E. coli. In order to identify the part of the protein that is necessary and sufficient for the recognition of haptoglobin, GST-tagged deletion mutant LPXTGp5 proteins were generated, as described previously. The homologous domains were attractive candidates for ligand binding. Using hyperimmune rabbit sera generated by immunisation with D1 and D2 proteins revealed cell surface location of both of these domains, demonstrating that they are available for extracellular ligand binding. Affinity chromatography was repeated with recombinant domain proteins and purified haptoglobin or IgG depleted plasma. GST-D1 and GST-D2 proteins were immobilised on Glutathione Sepharose column and human plasma or purified haptoglobin were applied as ligand sources, similarly how it was used for the full-length LPXTGp5. 2D analysis of the eluates identified haptoglobin as binding partner for the domains. Although this experimental setup is not suitable for affinity measurements, it seemed, that GST-D1 bound better than GST-D2.

### Example 3

### Regulation of Expression of LPXTGp5/SAHpR

3/A. Preferential Expression in Stress Medium

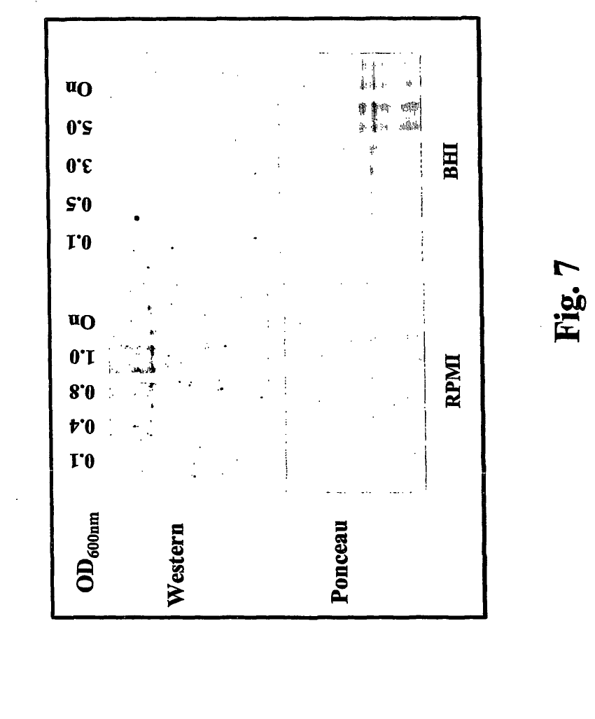

Immunoblot analysis of bacterial extracts prepared from S. aureus 8325-4 spa-(Protein A deficient strain) with anti-LPXTGp5 antibodies revealed a protein band with an approximately 130-kDa molecular weight (FIG. 7). Importantly, both the purified human IgGs and the rabbit immune sera revealed the same band. Although the predicted molecular weight of the 895 aa protein is 101-kDa, it is common for bacterial cell wall proteins to migrate slower than their actual size. In support of this notion, the recombinant full length LPXTGp5 indeed migrated as an ˜130-kDa protein similarly to the natural form (FIG. 2).

Interestingly, extracts prepared from bacteria grown in defined, poor and low-iron medium (RPMI 1640), but not from bacteria grown in rich medium, such as brain-heart-infusion (BHI) contained LPXTGp5 (FIG. 7). Moreover, expression of the protein was observed only in late log and stationary phase, but not in the early logarithmic phase of bacterial growth. It is known that defined, poor media with ion concentrations similar to human plasma force the pathogens to express more proteins with in vivo relevance relative to rich media, routinely used for laboratory growth of bacteria.

3/B. Regulation of Expression by Iron and Fur

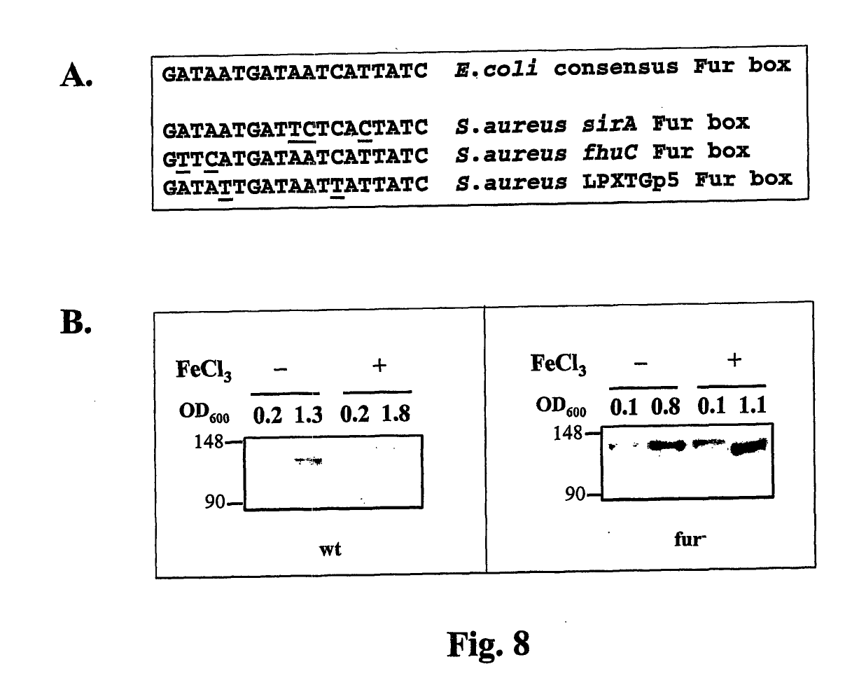

Nucleotide sequences upstream of LPXTGp5 ORF correspond to consensus fur binding box between −53 and −35 bps upstream from the starting ATG codon (FIG. 8A). The presence of this DNA sequence motif is highly predictive for iron dependent repression of expression, as it has been shown for several genes in both Gram− and Gram+ bacteria (Escolar et al, 1999).

The presence of fur box sequences in the gene evoked an interest in comparing expression of LPXTGp5 protein in bacteria grown in low and high iron concentration media. Immunoblot analysis of bacterial lysates prepared from 8325-4 wt S. aureus strains grown in iron supplemented RPMI medium revealed that iron suppresses expression to an undetectable level (FIG. 8B left panel). This regulation was lost in a S. aureus strain, in which the fur gene is inactivated by insertion mutagenesis. Fur is an iron concentration dependent transcriptional repressor protein, thus in its absence, target genes are expected to be relieved from repression and being upregulated. Indeed, LPXTGp5 was constitutively expressed in fur deletion mutant S. aureus strain (with 8325-4 background) in every growth phases (FIG. 8B right panel). Based on these results, it is established that expression of LPXTGp5 is under iron regulation mediated by Fur.

In contrast, agr and sar, key loci involved in the regulation of numerous staphylococcal virulence proteins (Arvidson and Tegmark, 2001) do not seem to regulate expression of LPXTGp5, based on results with extracts from single knock-out strains (agr-, sar-) showing similar staining intensity of the 130-kDa band relative to the those obtained with the wild type strain (data not shown).

### Example 4

### Assays for Screening and Development of Inhibitors of LPXTGp5-Haptoglobin Interaction

4/A. In Vitro, ELISA Based Assay Using Recombinant LPXTGP5 Proteins

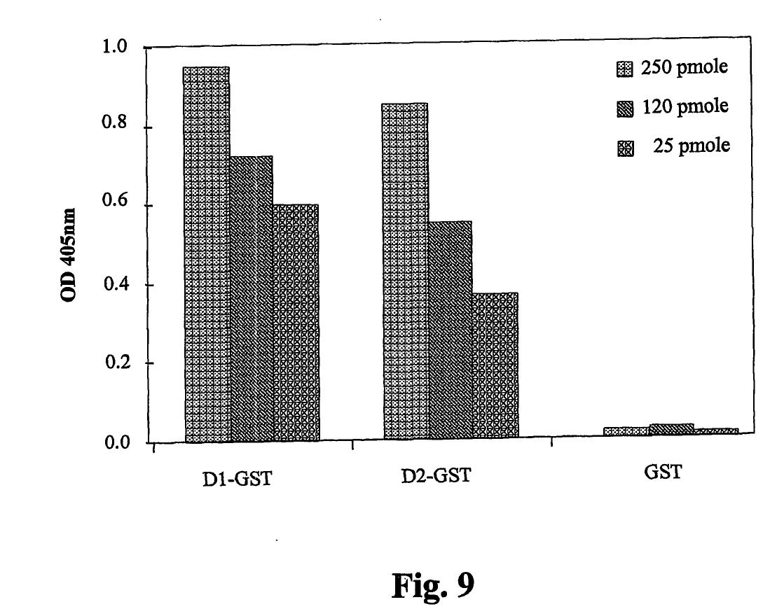

An ELISA based assay was developed using haptoglobin as a coating reagent (10 μg/ml in coating buffer) and GST-D1 and GST-D2 as binding partners. Hp-D1 and -D2 interactions were detected with biotin-labelled anti-GST mAbs (cat# ab6648, Abcam) and Streptavidin-HRPO (cat# 1089153, Roche). Increasing amount of D1 and D2 resulted in higher OD readings, until it reached saturation levels at about 250 pmole. The same molar amounts (corrected for actual molecular weights) of GST did not result in detectable signal relative to the background (FIG. 9). These results further supported the localisation of Hp binding within LPXTGp5. It also provides an easy, potentially high through put assay for screening inhibitors of LPXTGp5 and Hp interaction.

4/B. In Vitro, FACS Based Assay Using Living S. aureus Cells

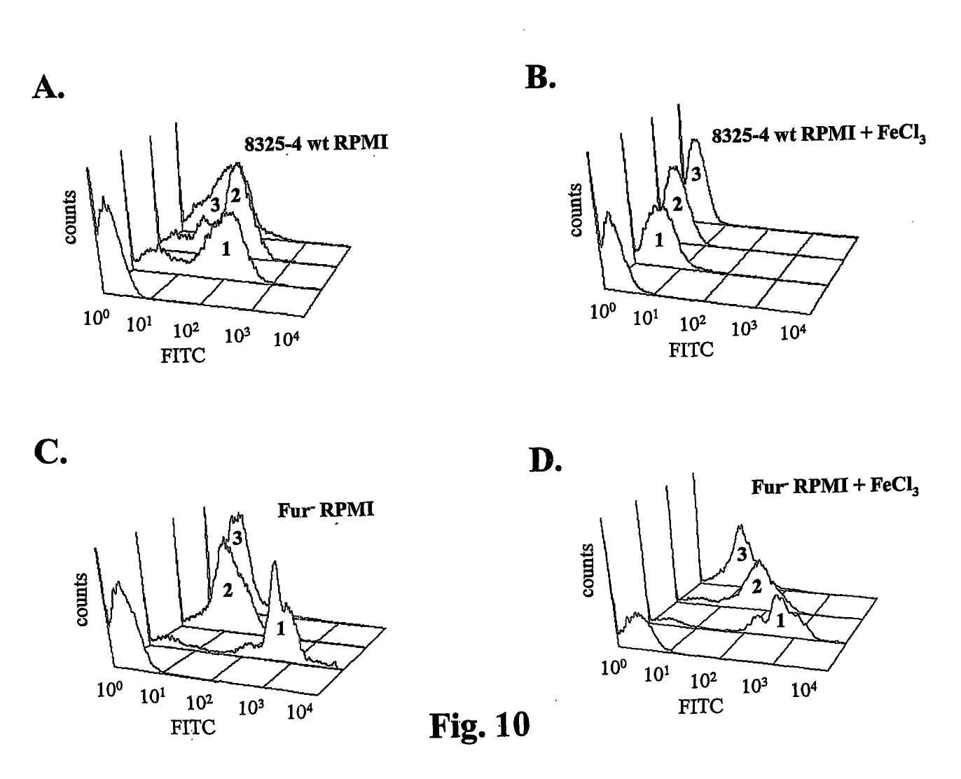

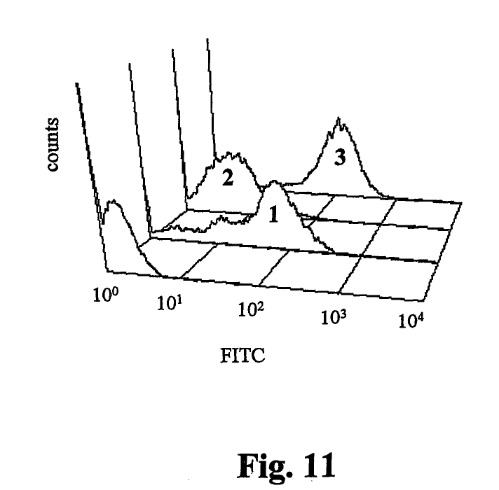

Purified haptoglobin was labelled with biotin (10:1 biotin to haptoglobin ratio) and added at increasing concentration to living wt and fur mutant S. aureus cells grown in RPMI medium in the absence or presence of 25 μM FeCl₃, as iron source. Haptoglobin binding was detected by using Streptavidin-FITC (cat# F0422, DAKO) as a secondary reagent and analysis was quantified by FACS. Surface staining of S. aureus grown under conditions, which allowed for the expression of LPXTGp5, demonstrated significant binding of haptoglobin (FIG. 10A). Haptoglobin binding was not detectable with S. aureus grown in iron-replete medium (FIG. 10B), which was consistent with the lack of signal on immunoblots with anti-LPXTGp5 antibodies. Importantly, fur mutant overexpressing LPXTGp5 irrespective of the iron concentration in the environment showed the most pronounced haptoglobin binding (FIG. 10C, D). To test the specificity of ligand binding to LPX-TGp5, haptoglobin was also added in the presence of recombinant GST-D1 domain. Increasing molar excess of GST-D1 decreased the surface staining achieved with haptoglobin in a concentration dependent manner. Nine times excess of GST-D1 already reduced haptoglobin binding to almost background level, while the same molar excess of GST did not affect binding significantly (FIG. 11). This competition experiment strongly suggests that there is a single staphylococcal haptoglobin receptor, and haptoglobin binding function is not redundant. Consequently, disruptive agents, whether antibodies, mimotopes or small drugs interrupting haptoglobin-LPXTGp5 interaction are likely to be deleterious for Hp-S. aureus interactions.

### Example 5

### Utilisation of Iron from Hp-Hb Complexes by S. aureus

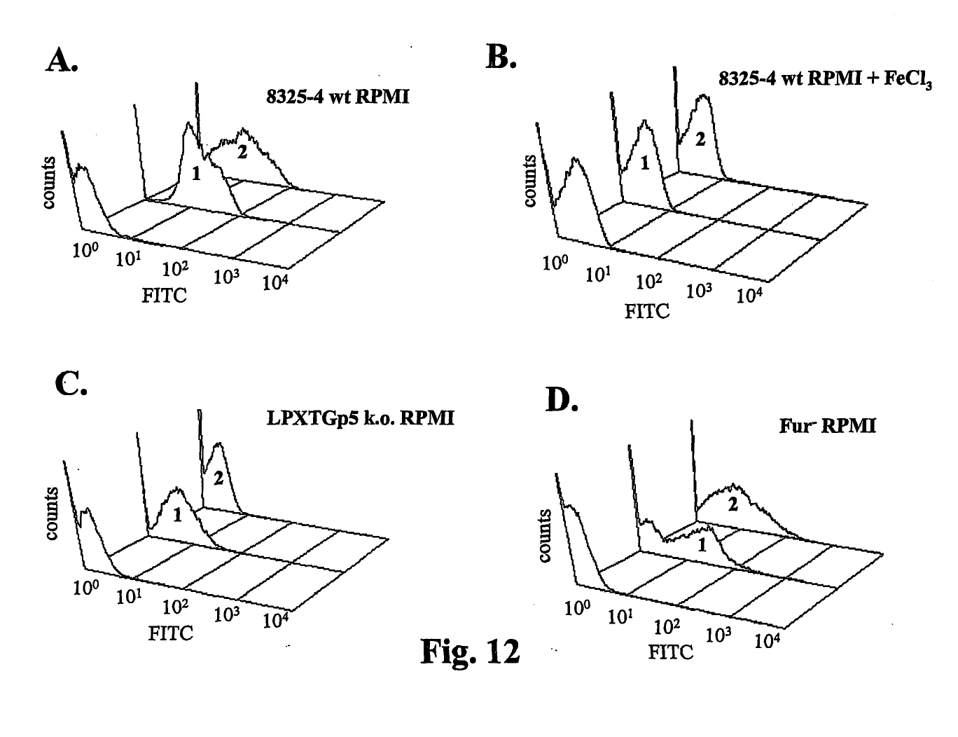

S. aureus growing inside the human body has to ‘steel’ iron from the body, namely from host iron binding proteins. The most abundant iron-containing protein is hemoglobin (Hb). Since extracellular hemoglobin is immediately complexed by haptoglobin, in practice it is the Hp-Hb complex, which serves as one of the sources of iron in the plasma. S. aureus 8325-4 strain grown in iron depleted RPMI medium was tested for its ability to use iron from Hp-Hb complexes in vitro. As negative control, only haptoglobin and only hemoglobin was added, and FeCl₃ as positive control for growth enhancement. S. aureus growth was stimulated in the presence of complexes, suggesting for acquisition of iron through Hp-Hb complexes (FIG. 12).

### Example 6

### Construction of an LPXTGp5 Insertionally Inactivated Mutant

To study the role of LPXTGp5 an insertionally inactivated LPXTG-p5 mutant was made. A plasmid for disrupting LPXTGp5 was constructed by PCR amplification of 1 kb 5′ and 3′ flanking regions of the LPXTGp5 open reading frame using gene specific primers with added SalI, KpnI, KpnI and EcoRI restriction sites, respectively on the primers. PCR products were cut with SalI-KpnI and KpnI-EcoRI and cloned into pAUL-A vector cut with SalI-EcoRI to give plasmid PAUL-AD in E. coli DH10B. A 1.5 kb tetracycline resistence cassette was amplified from pDG1513 using MOL1317 and MOL1318 primers, with incorporated KpnI restriction sites. A KpnI fragment containing a tetracyclin resistance cassette was dephosphorylated, and cloned into dephosphorylated KpnI site in pAUL-AD to give pAD02 in E. coli DH10B. Plasmid DNA of pAD02 (50 μg) was transformed into S. aureus RN4220 by electroporation and erythromycin-resistant transformants were identified at the permissive temperature for plasmid replication (30° C.). Single crossover Campbell-type chromosomal insertions were selected by shifting temperature to 42° C. while selecting on tetracycline. The presence of an inactivated and an intact copy of LPXTGp5 in clone 02 were verified by PCR using LPXTGp5 internal primers. A phage lysate of 02 was prepared from Φ11 stocks, transduced into S. aureus 8325-4 and selected on tetracycline plates. All of the recovered transductants were tested for erytromycin and lincomycin sensitivity. Successful integration of the LPXTGp5::Tc marker into the S. aureus chromosome of one of these transductants 47 was confirmed by PCR using LPXTGp5 internal primers and Southern blot analysis with the tetracycline and LPXTGp5 N-terminal fragment as the probe. Southern blot was performed according to standard procedure, and signal was developed with DNA probes prepared by PCR DIG Probe Synthesis Kit (Roche), according to the manufacturer's instructions. Briefly, after transfer, the membranes were prehybridised and hybridised under high stringency conditions (DIG Easy Hyb Solution at 42° C.). Washing was done twice with Low Stringency Buffer (2×SSC+0.1% SDS) and twice with High Stringency buffer (0.5×SSC+0.1% SDS). These hybridisation conditions allow the detection of genes having >80% homology with the probe according to the manufacturer's manual provided with the kit. The Southern blot analysis confirmed the presence of the LPXTGp5 gene in the wild type strain, and its absence in the knock-out strain. Under these conditions, the probe also detected a band, which corresponded to LPXTGp6, and were present in both strains.

### Example 7

### HarA Preferentially Binds to Haptoglobin-Haemoglobin Complexes

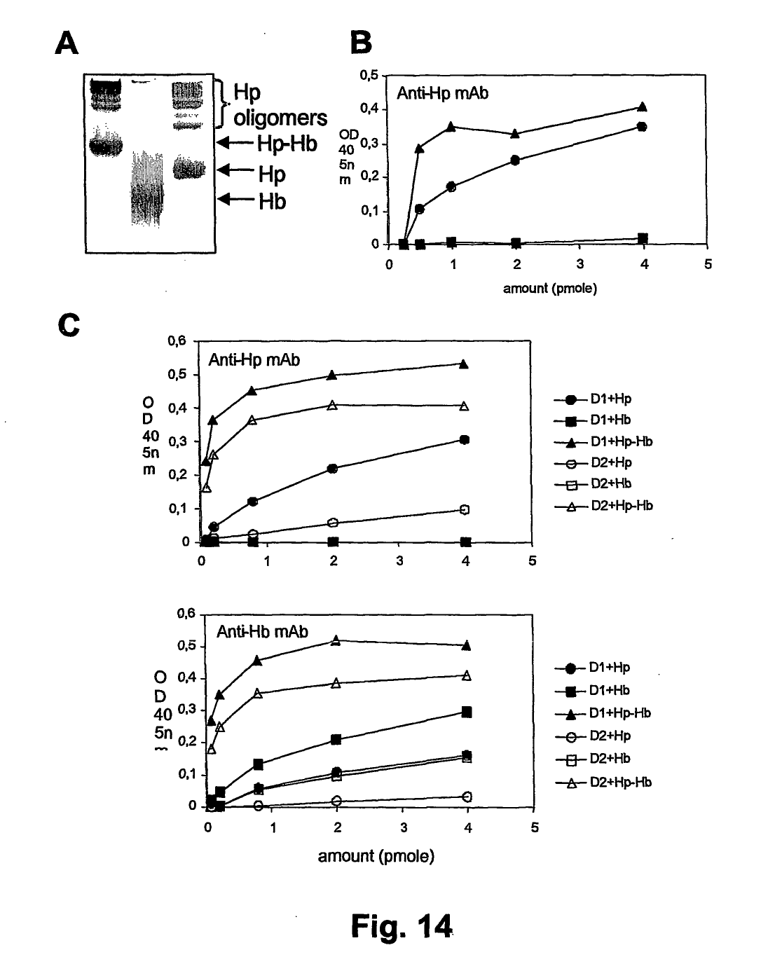

The main physiological role of haptoglobin is to complex extracellular haemoglobin in the plasma. Given the extremely high affinity of this interaction, capturing of released haemoglobin by haptoglobin is almost instantaneous. To address the question whether HarA can recognize haptoglobin as a ligand when it is bound to haemoglobin, we performed in vitro binding studies using rHarA, as well as HarA-D1 and HarA-D2 domain proteins. In these assays proteins were immobilized by coating ELISA plates, then haptoglobin, haptoglobin-haemoglobin complexes and also haemoglobin were added in increasing amounts and signal was detected by anti-haptoglobin or anti-haemoglobin monoclonal antibodies. Efficient complex formation between haptoglobin and haemoglobin was confirmed by native gel analysis (FIG. 14A). By comparing signal intensities generated with the different ligands at the same molar quantities, we detected a significantly higher binding of haptoglobin-haemoglobin complexes relative to haptoglobin. The higher level of binding at low ligand concentrations was suggestive for higher affinity interactions and was evident with both the full length HarA, as well as with the domain proteins (FIGS. 14B and C upper panel). Importantly, increased binding of the complexes was not the result of a high affinity interaction of HarA domains with haemoglobin, although we could demonstrate direct binding of Hb as well (FIG. 14C lower panel). The very same results were obtained in binding assays when we used the ligand proteins for coating and the GST tagged domain proteins for detection (data not shown). These data suggest that both haptoglobin and haemoglobin can be recognized by HarA domains as binding partners with a lower affinity compared to Hp-Hb complexes. However, given the very low concentration of free haemoglobin in the plasma, the physiologically relevant interaction seems to be between HarA and haptoglobin-haemoglobin complexes, as well with the abundant haptoglobin.

Figure Legends

FIG. 1 (A) LPXTGp5 is a typical Gram positive cell wall protein consisting of signal peptide (SP) on the N-terminus, extracellular domain and LPXTG cell sorting signal on the C-terminus, followed by a hydrophobic transmembrane domain (TM) and positively charged tail (++). Within extracellular part of the protein two highly homologues domains (D1, D2) were identified. (B) Similarity in secondary structure between LPXTGp5 /SA1552/ domains and other S. aureus proteins: LPXTGp6 /SA0976/, and LPXTGp7 /SA0977/ are shown.

FIG. 2 (A) Coomassie Blue stained 10% SDS-PAGE gel of recombinant LPXTGp5. Lane 1—molecular weight marker, lane 2—BSA (2mg/mL), lane 3—BSA (1 mg/mL), lane 4—LPXTGp5. (B) Immunoblot of recombinant LPXTGp5 with isolated anti-LPXTGp5 antibodies. (C) Human serum anti-LPXTGp5 antibody titers measured in ELISA (upper panel) are compared with immunoblot signal with rLPXTGp5 (lower panel).

FIG. 3 Anti-LPXTGp5 IgG titers determined in a standard ELISA in healthy donors (closed grey circles) and patients infected with S. aureus (blood infections—opened diamonds, wound infections—closed square, other infections—closed triangle).

FIG. 4 IgG antibody titers against D1 (A) and D2 (B) measured in a standard ELISA in healthy non-carrier (closed grey circles), in nasal carriers (closed black circles) and patients infected with S. aureus (blood infections—opened diamonds, wound infections—closed square, other infections—closed triangle).

FIG. 5 Coomassie Blue stained 2D electrophoresis gels. IgG depleted human plasma was bound to 20 μg recombinant LPXTGp5 protein. Specific binding partners were eluted with 100 μl sample buffer—9M Urea, 4% CHAPS, 100 mM DTT, 0.5% SDS. Plasma proteins binding to LPXTGp5 coupled to Stretpactin agarose (A), Streptactin agarose alone (B), purified StrepII-tagged rLPXTGp5 (C). IgG depleted human plasma (D).

FIG. 6 S. aureus lysate from 8325-4 spa-cells grown in RPMI to exponential phase was applied on an affinity column prepared by immobilising biotin-labelled haptoglobin on Streptavidin matrix. Nonspecific binding of lysate proteins to Streptavidin beads was considered as a background (lane 4). Immunoblot using human anti-LPXTGp5 antibody showed that native LPXTGp5, eluted from Hp-Streptavidin column (lane 3) is a binding partner for haptoglobin. As a positive control for immunoblot, recombinant LPXTG-p5 (lane 1) and S. aureus 8325-4 spa-lysate (lane 2) was used.

FIG. 7 S. aureus wild type (8325-4) strain was grown either in RPMI 1640 or Brain Heart Infusion (BHI) medium and grown till OD600 nm indicated. Total bacterial lysates were prepared using lysostaphin digestion and sonication. 20 μg of total protein was loaded on 7.5% polyacrylamide gel. Electophoreticaly separeted proteins were transferred to Hybond ECL membrane using semidry system. Membrane was probed with affinity purified human anti-LPXTGp5 IgG and the signal was developed using ECL detection system.

FIG. 8 (A) Comparison of known Fur box nucleotide sequences with a putative Fur box located upstream of LPXTGp5 gene. (B) Immunoblot analysis of S. aureus total lysate from wild type 8325-4 (wt) and fur mutant (fur-) strains after growth in different media (RPMI, RPMI+FeCl₃). 10 μg of total protein was loaded on a 7.5% polyacrylamid gel. Electophoreticaly separated proteins were transferred to ECL membrane using semidry system. Membrane was probed with affinity purified anti-LPXTGp5 IgG and the signal was developed using ECL detection system.

FIG. 9 Haptoglobin binding to GST-D1 and GST-D2 was performed in ELISA based assay. Polysorb ELISA plate was coated with haptoglobin o/n, and then GST-D1, GST-D2 and GST alone as a negative control were added in increasing concentrations. Specific signal was developed by using biotin-labelled anti-GST mAbs and Streptavidin-HRPO.

FIG. 10 Haptoglobin binding to S. aureus cells was detected in a FACS based assay. Biotin-labelled Hp (30 μg, 12.5 μg, 5 μg) was incubated for 30 min at RT with 5×10⁶ S. aureus wt 8325-4 strain (A, B) or fur- (C, D) grown in RPMI (A, C) or RPMI supplemented with 25 μM FeCl₃ (B, D). After washing Streptavidn-FITC was added for 30 min at RT, then cells were fixed with 2% Pfa and samples were analysed on FACScan. Fluorescence intensity of control cells (grey) was compared with fluorescence of cells bound to 30 μg Hp (1), 12.5 μg Hp (2) and 5 μg Hp (3).

FIG. 11 Haptoglobin binding to S. aureus cells was detected in a FACS based assay. 12.5 μg of biotin-labelled Hp alone (1), or complexed with 9× molar excess of D1-GST (2) or with GST (2) was incubated for 30 min at RT with 5×10⁶ S. aureus cells (wt 8325-4 strain) grown in RPMI. After washing Streptavidn-FITC was added for 30 min at RT, then cells were fixed with 2% Pfa and samples were analysed on FACScan.

FIG. 12 Haptoglobin binding to S. aureus 8325-4 wild type stain (wt), LPXTGp5 knockout stain (LPXTGp5 KO) was compared in a FACS based assay. Biotin-labelled Hp (20 μg, 5 μg) was incubated for 30 min at RT with 5×10⁶ S. aureus wt 8325-4 strain (A, B), LPXTGp5 KO. (C) or fur- (D) grown in RPMI (A, C, D) or RPMI supplemented with 25 μM FeCl₃ (B). After washing Streptavidn-FITC was added for 30 min at RT, then cells were fixed with 2% Pfa and samples were analysed on FACScan. Fluorescence intensity of control cells (grey) was compared with fluorescence of cells bound to 20 μg Hp (1) and 5 μg Hp (2).

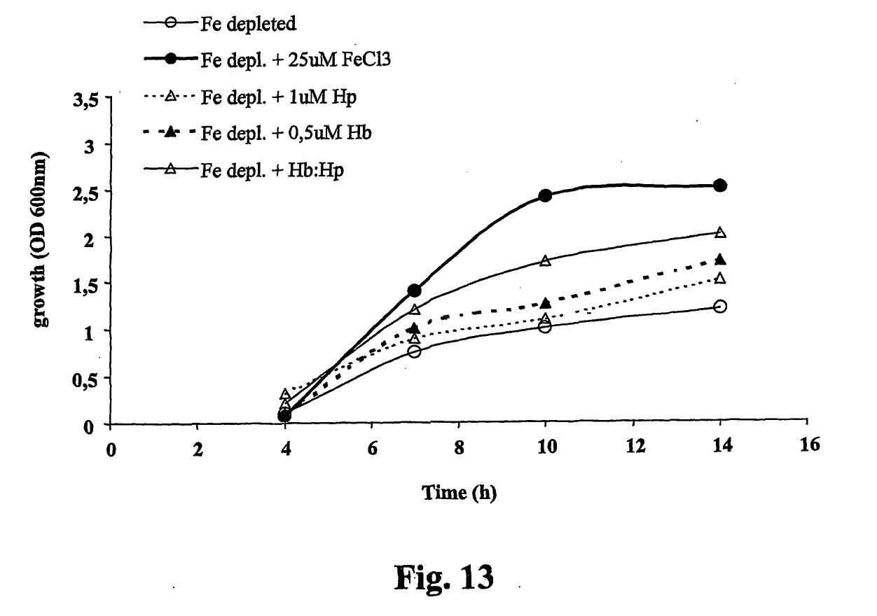

FIG. 13 Growth rate in media containing different iron sources. S. aureus wt 8325-4 cells were grown in iron depleted RPMI medium (open circle) or resupplemented with 25 mM FeCl₃ (closed circle), with 1 mM Hp (open triangle, dotted line), with 0.5 mM Hb (closed triangle, dotted line) and with Hp:Hb complexes (open triangle, continuous line). At indicated time points OD_{600 nm} of bacterial cultures was measured.

FIG. 14 HarA binds haptoglobin-haemoglobin complexes. A. Haptoglobin-haemoglobin complexes (Hp-Hb) were formed by incubation of haptoglobin (Hp) and haemoglobin (Hb) at a 1:1 molar ratio, and visualized by CBB stained native PAGE gels. B. Binding of purified haptoglobin (●) and haemoglobin (▪) was compared to that of haptoglobin-haemoglobin complexes (▴) in an ELISA based assay, using recombinant HarA as coating reagent and anti-haptoglobin mAb as detection reagent. C. GST-D1 (filled symbols) and GST-D2 (open symbols) proteins were coated on ELISA plates, and Hp, Hb and Hp-Hb complexes (as indicated in B) were added at increasing amounts. Signal was developed with monoclonal anti-haptoglobin (upper panel) or antihaemoglobin antibodies (lower panel).

### REFERENCES

Arvidson et al, (2001) Int J Med Microbiol 291: 159-170.
Baumann et al, (1990) Mol Cell Biol 10: 5967-5976.
Berkova et al, (1999) J Immunol 162: 6226-6232.
Biswas et al, (1995) Infect Immun 63: 2958-2967.
Bonnah et al, (1995) Microb Pathog 19: 285-297.
Borezee et at, (2000) J Bacteriol 182: 5931-5934.
Braun, (2001) Int J Med Microbiol 291: 67-79.
Cossley, ed. (1997) Staphylococci in health and disease.
Cornelissen et al, (1992) J Bacteriol 174: 5788-5797.
Delanghe et al, (1998a) AIDS 12: 1027-1032.
Delanghe et al, (1998b) Clin Chem Lab Med 36: 691-696.
El Ghmati et al, (1996) J Immunol 156: 2542-2552.
Escolar et al, (1999) J Bacteriol 181: 6223-6229.
Etz et al, (2002) Proc Natl Acad Sci USA 99: 6573-6578.
Gray-Owen et al, (1995) Infect Immun 63: 1201-1210.
Greenberg et al, (1989) Infect Immun 57: 1113-1118.
Gresham et al, (2000) J Immunol 164: 3713-3722.
Hochberg et al, (2002) Atherosclerosis 161: 441-446.
Horsburgh et al, (2001) Infect Immun 69: 3744-3754.
Jin et al, (1996) Infect Immun 64: 3134-3141.
Jonsson et al, (1991) Eur J Biochem 202: 1041-1048.
Josefsson et al, (2001) J Infect Dis 184: 1572-1580.
Kahler et al, (2001) Infect Immun 69: 1687-1696.
Kristiansen et al, (2001) Nature 409: 198-201.
Lewis et al, (1997) Mol Microbiol 23: 737-749.
Lounis et al, (2001) J Clin Virol 20: 123-126.
Mazmanian et al, (2002) Proc Natl Acad Sci USA 99: 2293-2298.
McDevitt et al, (1997) Eur J Biochem 247: 416-424.
Modun et al, (1999) Infect Immun 67: 1086-1092.
Ni Eidhin et al, (1998) Mol Microbiol 30: 245-257.
Nilsson et al, (1998) J Clin Invest 101: 2640-2649.
Patti et al, (1994) Annu Rev Microbiol 48: 585-617.
Patti et al, (1992) J Biol Chem 267: 4766-4772.
Pettersson et al, (1994) Microb Pathog 17: 395-408.
Ren et al, (1998) Infect Immun 66: 4733-4741.
Rennermalm et al, (2001) Vaccine 19: 3376-3383.
Rossbacher et al, (1999) Scand J Immunol 50: 399-404.
Schneewind et al, (1995) Science 268: 103-106.
Schryvers, (1988) Mol Microbiol 2: 467-472.
Sebulsky et al, (2001) J Bacteriol 183: 4994-5000.
Sebulsky et al, (2000) J Bacteriol 182: 4394-4400.
Smith et al, (1995) Science 268: 284-286.
Stojiljkovic et al, (1996) J Bacteriol 178: 4670-4678.
Taylor et al, (2002) Mol Microbil 43: 1603-1614.
Ton-That et al, (1999) Proc Natl Acad Sci USA 96: 12424-12429.
Van Vlierberghe et al, (2001) Eur J Gastroenterol Hepatol 13: 1077-1081.
Vytvytska et al, (2002) Proteomics 2: 580-590.
Wagner et al, (1996) J Immunol 156: 1989-1996.
Webb et al, (1999) Infect Immun 67: 2138-2144.
Yang et al, (2000) Am J Respir Cell Mol Biol 23: 277-282.

1-17. (canceled)

18. A method of treating or preventing Staphylococcus infection comprising:
obtaining a molecule that interacts with the haptoglobin receptor ligand; and
administering the molecule to a subject.

19. The method of claim 18, wherein the subject is a human.

20. The method of claim 18, wherein the molecule disrupts the haptoglobin receptor ligand binding.

21. The method of claim 18, wherein the molecule is a haptoglobin receptor antibody or a haptoglobin mimotope that binds to a peptide of SEQ ID NO:2 or haptoglobin receptor or a fragment thereof.

22. The method of claim 21, wherein the haptoglobin receptor antibody or haptoglobin mimotope binds to a peptide of SEQ ID NO:4 or SEQ ID NO:6 or a fragment thereof.

23. The method of claim 18, wherein the molecule is an anti-sense nucleic acid that binds to a haptoglobin receptor gene or to a regulatory element for the expression of the haptoglobin receptor gene.

24. The method of claim 23, wherein the anti-sense nucleic acid binds to the Fur box of SEQ ID NO:7.

25. The method of claim 23, wherein the molecule is an inhibitory compound that binds to the Fur box of SEQ ID NO:7.

26. The method of claim 23, wherein the molecule is a nucleic acid molecule capable of hybridizing under stringent conditions to a nucleic acid molecule having the sequence of SEQ ID NO:7.

27. An isolated polynucleotide comprising a nucleic acid sequence of SEQ ID NO:3 or SEQ ID. NO:5.

28. An isolated polypeptide comprising a polypeptide sequence of SEQ ID NO:4 or SEQ ID NO:6.

29. A synthetic conjugate comprising a peptide of SEQ ID NO:4 linked by a non-naturally occurring linker to a peptide of SEQ ID NO:6.

30. The synthetic conjugate of claim 29, wherein the non-naturally occurring linker is a polypeptide.

31. A process for isolating molecules that interact with a haptoglobin receptor ligand binding comprising:
providing a haptoglobin receptor polypeptide or haptoglobin binding fragment thereof on a solid surface;
binding labeled haptoglobin to the immobilized haptoglobin receptor polypeptide or haptoglobin binding fragment thereof to form a complex between immobilized haptoglobin receptor polypeptide or haptoglobin binding fragment thereof and labeled haptoglobin;
contacting the complex with a pool containing at least one candidate molecule;
determining whether the at least one candidate molecule replaces the labeled haptoglobin in the complex; and
isolating any molecules replacing the labeled haptoglobin in the complex.

32. The process of claim 31, wherein the haptoglobin binding fragment is of SEQ ID NO:4, SEQ ID NO:6, a fragment thereof or combination of fragments thereof.

33. The process of claim 31, wherein the haptoglobin receptor is S. aureus haptoglobin receptor.

34. The process of claim 31, wherein the haptoglobin is human haptoglobin.

35. A process for isolating molecules that interact with a haptoglobin receptor ligand binding comprising:
providing a haptoglobin immobilized on a solid surface;
binding at least one labeled haptoglobin receptor polypeptide or haptoglobin binding fragment thereof to the immobilized haptoglobin to form a complex between immobilized haptoglobin and the labeled haptoglobin receptor polypeptide or haptoglobin binding fragment thereof;
contacting the complex with a pool containing at least one candidate molecule;
determining whether the at least one candidate molecule replaces the labeled haptoglobin receptor polypeptide or haptoglobin binding fragment thereof in the complex, and binds to the immobilized haptoglobin; and
isolating any molecules of the pool bound to the immobilized haptoglobin.

36. The process of claim 35, wherein the haptoglobin binding fragment is of SEQ ID NO:4, SEQ ID NO:6, a fragment thereof or combination of fragments thereof.

37. The process of claim 35, wherein the haptoglobin receptor is S. aureus haptoglobin receptor.

38. The process of claim 35, wherein the haptoglobin is human haptoglobin.

39. A process for isolating molecules that interact with a haptoglobin receptor ligand binding comprising:
providing a pool of candidate molecules,
removing and isolating from the pool molecules that bind to immobilized haptoglobin receptor or haptoglobin binding fragments thereof,
removing and isolating from the pool any molecules which bind to immobilized haptoglobin,
contacting the remaining pool of candidate molecules with an immobilized complex formed between haptoglobins and haptoglobin receptors or haptoglobin binding fragments thereof; and
isolating the molecules which bind to the immobilized complex.

40. The process of claim 39, wherein the haptoglobin binding fragment is of SEQ ID NO:4, SEQ ID NO:6, a fragment thereof or combination of fragments thereof.

41. The process of claim 39, wherein the haptoglobin receptor is S. aureus haptoglobin receptor.

42. The process of claim 39, wherein the haptoglobin is human haptoglobin.
